(19) United States
(12) Patent Application Publication
(10) Pub. No.: US 2004/0110251 A1
(43) Pub. Date: Jun. 10, 2004

(54) Detection of pathogenic bacteria

(76) Inventors: Reiner Grabowski, Gottingen (DE); Cordt Groenewald, Berlin (DE); Astrid Schneider, Berlin (DE); Andreas Pardigol, Frankreich (DE); Kornelia Berghof, Berlin (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/250,997

(22) PCT Filed: Oct. 15, 2001
(86) PCT No.: PCT/EP01/11901

(30) Foreign Application Priority Data
Jan. 8, 2001 (DE) 10100493.1

(51) Int. Cl. C12P 21/06

(52) U.S. Cl. 435/69.1

(57) ABSTRACT

The object of the invention are oligonucleotides which can be used for the detection of pathogenic bacteria. With the aid of these oligonucleotides a method can be applied which enables the selection of pathogenic bacteria from non-pathogenic bacteria. Preferably the detection of the bacteria includes a PCR. Oligonucleotides are also provided which can be used as a positive control for the PCR.

This invention relates to a method for the detection of EHEC bacteria and to oligonucleotides suitable for this detection.

In the age of international transport and rational processing methods the importance of pathogenic bacteria transmitted through foodstuffs is growing. Often raw materials from many different parts of the country are brought together at a central point, mixed thoroughly and processed to form a certain foodstuff. If one of the raw products was the carrier of a pathogenic germ, then it can reproduce during the production process and lead to the contamination of a large batch of foodstuff.

In this connection Escherichia coli has arisen as a very important pathogenic germ. Following campylobacter and salmonella, it is the third most common germ contaminating foodstuffs. The bacterium normally occurs as a harmless commensal in the human intestine. However, it can take up certain pathogenicity genes and can then represent a fatal risk. Consequently, a whole series of E. coli sub-types have been characterised which have high pathogenic potential. These include the Shigella strains which are really to be grouped systematically under E. coli. Also worth mentioning are EPEC (enteropathogenic E. coli) which in particular cause diarrhoea illnesses with newborn/infants, ETEC (enterotoxinogenic E. coli), which form extracellular thermally stable and thermally unstable toxins and are mainly responsible for travelling diarrhoea and EIEC, which penetrate the cells of the intestinal mucosa and cause bacillary dysentery.

An especially dangerous group of pathogenic E. coli strains are the EHECs (enterohemorrhagic E. coli). The group of EHECs also includes the particularly frequently occurring serotype O157:H7. This, as also the other members of the group, can cause the haemolytic-uraemic syndrome (HUS) which can be fatal. HUS is accompanied by diarrhoea containing blood and acute kidney failure.

The endemic occurrence of EHECs in nature is largely restricted to cattle, even if other sources, in particular pigs, have been documented as reservoirs. As a consequence, processed beef products, in particular minced meat, are often contaminated with EHECs. In some investigations into foodstuffs more than 50% of minced meat samples were positive to EHEC. In recent years other foodstuffs such as lettuce, radishes, milk and milk products have been identified as EHEC sources.

In the USA in the last few decades more than 20,000 E. coli O157:H7 infections occurred per annum (Boyce et al. 1995, N. Engl. J. Med. 333, 364-368), of which about 250 ended in death. However, the real figures may be much higher due to defective diagnosis. In Europe and Japan E. coli O157:H7 infections are primarily reported in summer. In contrast, in the southern hemisphere non-0157 EHEC serotypes are in particular of great importance.

The pathogenic potential of an EHEC strain is determined by its pathogenicity factors. Consequently, the occurrence of Sit genes (Shiga-like toxin or vtx=verotoxin gene) is a necessary, but not a sufficient prerequisite for pathogenicity. In addition, other factors have been characterised (Nataro and Kaper 1998, Clin. Microb. Rev. 11, 142-201), which are necessary to infect the host. Many of these factors are not constantly coded in the genome, but are rather located on transferable plasmides or in phage genomes. Therefore, the equipping of EHEC strains with pathogenicity factors may also be subject to chronological variability.

The reliable diagnostic detection of EHEC strains with known methods causes substantial problems. So microbiological methods are hardly suitable for obtaining reliable detection. Metabolic physiological differences between apathogenic E. coli and pathogenic EHEC strains are hardly present. The frequently characteristic defect of the uidA gene (beta-glucuronidase) for E. coli O157:H7 (Cebula et al. 1995, J. Clin. Microb. 33, 248-250) is not a reliable feature of the EHEC group. For this reason diagnostic methods must fall back on molecular biological features.

One of the methods frequently used in the past was serotyping by an ELISA. However, this presents many disadvantages, because it is relatively time-consuming and demands many working steps. In addition, its sensitivity is not sufficient for many diagnostic applications. Furthermore, the serotype alone is not a sufficient feature for pathogenicity.

Another method of differentiating between E. coli strains is to investigate differences in the DNA sequence. The technique is based in particular on the fact that pathogenic strains possess certain toxin genes. For example, the toxin genes similar to Shiga (Shiga-like toxins, sit or verotoxin genes, vtx) could be directly detected (Takeshi et al. 1997, Microb. Immun. 41, 819-822, Paton and Paton 1999, J. Clin. Microb. 37, 3362-3365). The PCR can be applied to amplify parts of the gene. These fragments can be rendered visible so that they act as a diagnostic characteristic.

The disadvantage of this method is that the sit genes are not a sufficient prerequisite for pathogenicity. Other DNA sequence features are necessary to establish an unambiguous correlation between the genotype and pathogenicity. The E. coli strains, which possess sit genes are designated VTECs (verotoxin forming E. coli or STECs). Consequently, they form a larger group than the EHECs.

Other genetic markers for EHEC or subgroups of it have also been tried out. These include the fimA gene (Li et al. 1997, Mol. Cell. Probes, 11, 397-406) and the fliC gene (Fields et al. 1997, J. Clin. Microb. 35, 1056-1070). However, they all have the disadvantage of mapping only part of the EHEC group.

Since the EHEC group does not form a systematic unit phylogenetically, there arises the difficult task of finding genetic polymorphisms through which it is unambiguously characterised. These polymorphisms should also be so reliable that they also acquire heterogeneities and genetic instabilities within the EHEC group. Apart from the specific detection, they should also permit the most sensitive detection of EHEC possible.

There are already some detection systems for E. coli classified as EHECs. Where they are based on immunological detection, their sensitivity is however not sufficient. In addition the detection of antibodies is very sensitive to external contaminations. Extracts from foodstuffs present significant problems, because they conceal the antigen surfaces of the bacteria or even destroy them. Where though some surface antigens reach exposure, they are often too few to ensure reliable detection with adequate sensitivity.

The object of this invention is to provide a method which ensures the reliable detection of EHEC bacteria in any sample and which is subject to the lowest possible impairment due to other constituents of the sample, such as PCR inhibitors, the DNA of non-pathogenic bacteria, or due to the quenching phenomenon (refer to the chapter “Optimisation of the on-line PCR”). Also, the object of the invention is to make the means required for EHEC detection available.

The first problem is solved according to the invention by a method for the detection of EHEC bacteria, incorporating the step of detection of the occurrence of a nucleic acid sequence from the Slt locus and/or eae locus and/or hlyA locus in the sample.

The second problem is solved according to the invention by an oligonucleotide selected from one of the nucleic acids including at least one sequence with one of the SEQ ID numbers 1-98 and/or derivatives of it.

Definitions

Fragments of Oligonucleotides

Fragments of oligonucleotides arise due to deletion of one or more nucleotides on the 5′ and/or 3′ end of an oligonucleotide.

Gene

The gene includes the open reading frame or coding area of a DNA. Also, the cistron is a gene which together with other cistrons is however located on one mRNA. DNA regions which regulate the transcriptions of the gene, such as the promoter, terminator, enhancer also belong to the gene.

Identical DNA Sequences/Percentage of Identity

For the determination of the identity (in the sense of complete matching, corresponding to 100% identity) of DNA or RNA sequences, partial sequences of a larger polynucleotide are considered. These partial sequences comprise ten nucleotides and are then identical when all 10 modules are identical for two comparative sequences. The nucleotides thymidine and uridine are identical. As partial sequences, all possible fragments of a larger polynucleotide can be considered.

As an example two polynucleotides are considered which comprise 20 nucleotides and which differ in the 5th module. In a sequence comparison six 10-way nucleotides are found which are identical and five which are not identical, because they differ in one module.

In addition, the identity can be gradually determined, whereby the unit is stated in percent. For the determination of the degree of identity partial sequences are also considered, which comprise as a minimum the length of the actually used sequence, e.g. as primer, or 20 nucleotides.

As an example, polynucleotide A with a length of 100 nucleotides and B with a length of 200 nucleotides are compared. A primer with a length of 14 nucleotides is derived from polynucleotide B. For the determination of the degree of identity, polynucleotide A is compared with the primer over its complete length. If the sequence of the primer occurs in polynucleotide A, whereby it however deviates in one module, then there is a fragment with a degree of identity of 13:14→92.3%.

In the second example the polynucleotides A and B previously mentioned are compared in their entirety. In this case all the possible comparative windows of a length of 20 nucleotides are applied and the degree of identity determined for them. If then nucleotides nos. 50-69 of polynucleotide A and B are identical with the exception of nucleotide no. 55, then a degree of identity of 19:20→95% arises for these fragments.

Multiplex PCR

A multiplex PCR is a Polymerase Chain Reaction or DNA or RNA amplification reaction in which more than two primers are used which are not regarded as a forwards-backwards primer pair. With the presence of all nucleotide target molecules to be detected, this leads to the creation of at least two different amplicons. These amplicons should at least differ in the region in which the primers link, but they can also be allocated to completely different genes. In the case of detection of the EHEC, the multiplex PCR, in the simultaneous detection of two or three genes, consists of the group SltI, SltII, eae and hlyA.

Nucleotides

Nucleotides are the modules of the DNA or RNA. The following abbreviations are used:

G=Guanosine, A=Adenosine, T=Thymidine, C=Cytidine, R=G or A, Y=C or T, K=G or T, W=A or T, S=C or G, M=A or C, B=C, G or T, D=A, G or T, H=A, C or T, V=A, C or G, N=A, C, G or T, I=Inosine.

On-line Detection

In relation to this invention, on-line detection is defined as the simultaneous running of two processes: the detection of the DNA or RNA and a process which leads to the provision of a detectable amount of DNA or RNA. With this process the release of genomic DNA/RNA from cells may, for example, be involved or the enrichment of DNA/RNA from a complex mixture or the amplification of polynucleotides, e.g. through a PCR. Detection is the perception of a signal which correlates to the presence and possibly the amount of the DNA/RNA. In the case of the PCR this type of signal may increase with the increasing amplification of the target DNA. On-line detection can be carried out also in a miniaturised form, e.g. on a chip. The signal can, for example, be produced through the fluorescent molecules of a probe, through radioactive molecules or through enzyme-coupled colour or fluorescence intensity.

The term on-line detection is synonymous to real-time detection.

Primer

Primers are oligonucleotides which act as starter molecules during a PCR. Here, they hybridise on a target molecule, which may be, for example, DNA or RNA, and are lengthened by a polymerase. They can also however act as probes.

Probe

Probes are oligonucleotides which hybridise on the target DNA or RNA molecules. They are used for the direct or indirect detection of these target DNA or RNA molecules. For this purpose, they can be coupled to fluorescent molecules or to molecules containing colouring agents. In addition they can be indirectly detected with an ELISA. In a special version they only produce a signal through FRET (Fluorescence Resonance Energy Transfer) when two probes hybridise adjacently in a defined manner. In this case a colouring agent on a probe is excited by a light beam and transfers its excitation energy to the colouring agent of the adjacent probe. This then emits light of a defined wavelength. They can also be used as primers.

EHEC and VTEC

EHECs are enterohemorrhagic E. coli and a subgroup of the VTEC. E. coli of the serotype O157 is a subgroup of the EHEC.

VTEC is characterised in that it either possesses the SltI (vtx1) or the SltII (vtx2) or both genes. EHECs are VTECs which also possess the eae gene and/or hlyA gene (coded for Intimine). In addition, they can be characterised by the presence of other pathogenicity genes such as hlyB, hlyC, fimA, fliC, etc.

Slt Locus

Slt locus signifies the locus containing the SltI gene or SltII gene, which are also designated as vtxI resp. vtxII. The nucleic acid sequence of this locus is known from the state of the art, for example from Paton, A. W. et al. 1995, Gene 153 (1), 71-74. The term “locus” as used in this connection comprises, apart from the coded region, also a section of 1000 nucleotides in each case on the 5′ end of the start codon or on the 3′ end of the stop codon.

Eae Locus and hlyA Locus

The sequences of the eae locus and the hlyA locus are also known from the state of the art, for example from Makino, K., et al. 1998, DNA Res. 5 (1), 1-9.

Derivatives of the Oligonucleotides According to the Invention

Derivatives of the oligonucleotides according to the invention are taken to mean sequences which differ in at least one nucleotide from the specific sequences according to SEQ ID numbers 1-98, for example, by at least one base interchange, an insertion, deletion or addition. These also include oligonucleotides which are at least 80% identical to one of the specific sequences according to SEQ ID numbers 1-98 and oligonucleotides with a comparable specificity of hybridisation. The latter signifies that the derivative produces the same hybridisation pattern with a specified sample containing nucleic acid, such as the oligonucleotide with one of the specific sequences with one of the SEQ ID numbers 1-98.

Biochip

Biochip is taken to mean carriers for the high throughput of analyses as marketed, for example, by AFFYMETRIX. The chips enable the testing of numerous different nucleic acids on one carrier.

The analysis of DNA exhibits substantial advantages compared to the serological detection, because there are standardised, simple purification methods for DNA analysis with which DNA can be separated from external matrices and purified further. Due to the size of the bacterial genome, selection can also take place from a substantial number of individual sequence motifs, whereas the selection of the previously mentioned exposed surface antigens is relatively low.

As sequences for the specific detection of EHEC bacteria, sequences from the Slt locus, the eae locus and the hlyA locus are suitable. Here, it is sufficient for the detection of EHEC in a specified sample if a partial sequence from the Slt locus and another of the quoted loci can be detected in the analysis sample. With the Slt locus two different gene loci are actually involved, SltI and SltII, whereby however only one of the two loci occurs with the numerous EHEC strains. The simultaneous detection of sequences from the Sit locus and the eae locus in a single sample provides sufficiently high proof. The simultaneous detection of a sequence from the Slt locus and the hlyA locus has a similar high reliability. A particularly high degree of reliability with regard to an EHEC contamination then arises if sequences from the three different loci, Slt, eae and hlyA, are simultaneously detected in one sample.

With another preferred embodiment the nucleic acid to be examined is passed to a PCR. This has the result that EHEC-specific amplicons are produced if nucleic acids of EHEC bacteria are present in the sample. Here in the simplest case, the PCR can be arranged as a simple linear PCR with only one oligonucleotide as primer, but preferably the PCR takes place however with so-called forwards and backwards primers for each genome section of the bacterial nucleic acid to be amplified.

With another preferred embodiment a primer combination is used whereby at least one primer is selected, comprising at least one sequence from one of the SEQ ID numbers 1-45 and 95-98, also designated as sequences of the categories A-C and a primer, comprising at least one sequence selected from one of the SEQ ID numbers 46-83 and 93 and 94, also designated as sequences of the categories D and E. According to the invention, derivatives of the mentioned primers can also be used for the detection. The derivatives normally lead to amplification of the same genome sections as indicated by the definitive primers according to the SEQ ID numbers 1-98.

With another preferred embodiment a primer pair consisting of a forwards primer and a backwards primer, selected from the category A-C, is used with a primer pair comprising a forwards primer and a backwards primer, selected from the category D and E. A preferred embodiment uses a primer pair from one of the categories A-C in combination with a primer pair from category D and another primer pair from category E.

With a further preferred embodiment the detection method includes the use of another primer comprising at least one sequence, selected from a sequence from category F. These sequences are characteristic of the genus E. coli. Consequently, for example, with a preferred strategy of EHEC detection, the analysis sample can be first analysed with a sequence selected from the category F. A positive result points to the presence of E. coli in the analysis sample. In a second step it can then be more closely determined, using the sequences from the categories A-E, whether the detected E. coli is a member of the EHEC group. The additional analysis with sequences from the category F can also occur of course as an additional measure after the analysis with the sequences from the categories A-E.

With a further preferred embodiment the various oligonucleotides and therefore the various PCR runs are carried out in the form of a multiplex PCR. Here, different amplicons are created in the PCR in a single initiated reaction with the aid of the various oligonucleotides. Alternatively, the multiplex PCR can also be subdivided to different PCRs, whereby a sequential train of PCRs is carried out, whereby each PCR is carried out with a specific primer or primer pair. In both cases, with the presence of EHEC bacteria in the analysis sample a band pattern is obtained indicating the presence of EHEC bacteria.

With a further preferred embodiment use is made of the so-called chip technology (biochips) in the detection method. Here, on one hand a large number of different analysis samples can be analysed on one chip in that the individual spots on the chip contain analysis material from different sources. On the other hand, the chip can carry a set of oligonucleotides, whereby each spot contains a specific oligonucleotide and this oligonucleotide pattern is brought into contact with analysis samples. In the case that the analysis material contains EHEC nucleic acid, it hybridises with the probes specific to the EHEC present on the chip and produces a corresponding signal pattern.

With a further preferred embodiment the detection method can include further steps, such as for example an amplification of the nucleic acid to be detected, whereby this preferably occurs using PCR and/or a southern hybridisation with EHEC-specific probes, whereby this hybridisation occurs without prior amplification or after amplification of the nucleic acid to be detected is concluded. Furthermore, the nucleic acid to be detected can be detected using the ligase chain reaction. Finally, the nucleic acid to be detected can be enriched by isothermal nucleic acid amplification.

With a further preferred embodiment, the amplification of the target nucleic acid can also take place using on-line detection.

With a further preferred embodiment the amplification of the nucleic acid to be detected and/or the detection of the contained amplicons occurs on a biochip, whereby it is particularly preferable to carry out the amplification and detection on one chip.

According to the invention, as a means for carrying out the method described above, oligonucleotides are selected from a nucleic acid, comprising at least one sequence with one of the SEQ ID numbers 1-98 or derivatives thereof. The stated oligonucleotides can on one hand be used as primers within the scope of a PCR and on the other hand also as probes, for example within the scope of a southern blot hybridisation. Depending on the requirements of the desired detection, the specialist can form the suitable combination of oligonucleotides as primers or probes.

With an especially preferred embodiment a combination of oligonucleotides is used, whereby at least one oligonucleotide is selected from sequences from the categories A -C and at least one oligonucleotide is selected from sequences from the categories D and E.

With another especially preferred embodiment the combination according to the invention furthermore comprises an oligonucleotide selected from the sequences of category F which are specific to the genus E. coli. Preferably, the stated oligonucleotides or combinations of them are used in the form of a kit for the detection of EHEC bacteria, whereby the kit also includes other reagents for the detection of bacteria or for conducting the detection reactions. In this respect, the reagents and enzymes required for the PCR and, where applicable, suitable carrier materials are also included, for example, such as is desired with the chip technology.

The oligonucleotides or oligonucleotide combinations according to the invention are therefore a suitable means for the specific and reliable detection of EHEC bacteria in any analysis samples.

With the invention of the polymerase chain reaction it is possible to amplify individual DNA polynucleotides and then to detect them with extremely high sensitivity. This technology opens up substantial new opportunities, but also exhibits new problems. For example, with the DNA amplification incorrect fragments can be easily amplified, leading to incorrect positive results in the analysis. In addition, it is very difficult to select the diagnostic DNA sequences characteristic to EHEC from the multitude of possibilities.

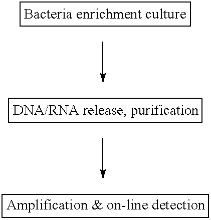

### Flowchart for the Detection of EHEC by PCR and Simultaneous Detection

This invention consists of a method and oligonucleotides which enable a qualitative and quantitative detection of EHEC. This method also includes a positive check for the PCR reaction which detects the genera of E. coli and Shigella. This is important, because with negative EHEC findings the correct sequence of the PCR reaction must be ensured. The detection method consists all together of four steps: propagation of the bacteria, purification of the DNA/RNA, amplification of the polynucleotides and detection of them. In a special method the two last steps can also take place simultaneously.

The propagation of the bacteria occurs in that the matrix to be investigated, e.g. a foodstuff or faecal sample is incubated with a currently available bacterial medium. Bacterial media are commercially available and can, for example, contain a proteolytically digested basic substance, such as soya broth, bile salts and a buffer such as dipotassium hydrogen phosphate. In addition, it is advantageous to add an inhibitor to the enriching medium which promotes the growth of the EHEC compared to other bacteria in the enrichment medium. Such inhibitors may be antibiotics, such as Novobiocin, for example.

In the second step the polynucleotides are purified. To do this, the bacteria are normally first separated from the medium by centrifuging and/or filtration. A further washing stage may follow. Then the bacteria are broken down. This takes place by heating, by an alkaline or acidic environment or by reagents which destabilise the bacteria cell wall, such as deionising chemicals or lysozyme. The genomic DNA or the RNA can now be directly used in a PCR reaction or it is purified further. For this purification materials are suitable on the surface of which the polynucleotides bond, e.g. positively charged surfaces or silicate surfaces. This material can be mounted in columns and is commercially available.

The PCR reaction and the detection of the amplicons represent the greatest importance in the detection of bacteria. As already explained, it is very difficult to find differences in DNA sequences between EHEC and other bacteria, in particular the harmless E. coli strains. A single PCR reaction with the amplification of a single DNA or RNA region alone would not appear to offer a very reliable foundation for marking the strain limits. A preferred element of the invention is that various regions of the EHEC genome can be amplified simultaneously and/or sequentially. Preferably, further DNA/RNA sequences are amplified in a consecutive step for the concluding analysis. If all significant amplicons can be detected simultaneously, e.g. on one chip, then the “first” amplification step and the “consecutive” amplification step can also run in a single PCR reaction or in a single PCR reaction vessel. The key to the application of the primers and probes is given below.

The system for the detection of EHEC makes primers available which optimally map the EHEC group in certain combinations. The detection is, for example, carried out in two independent PCR runs in primer multiplex arrangements. In a first run the primers and probes of categories A, B and/or C are employed. In the second run only the samples are used which were positive in the first run. In this second run the primers and probes of categories D and E are used. Within one category a forwards primer and a backwards primer can be combined with one another in each case. So multiplex PCRs are carried out in which many target DNA or RNA fragments are propagated simultaneously in one reaction. Due to this process a very differentiated picture of the bacterial populations present can be obtained. Depending on the practical requirements to the sensitivity of the EHEC detection and when the simultaneous detection is possible, all detection primers (for categories A+B+C and D or E and possibly category F) can also be used in a single multiplex PCR.

TABLE 1
Forwards primers, category A

| | |
|---|---|
| No. | Primer sequence |

| | |
|---|---|
| 1 | CTGGGGAAGGTTGAGTAG |
| 2 | GTCCTGCCTGAYTATCATGG |
| 3 | ACAAGACTCTGTTCGTGTAGG |
| 4 | AAGAATTTCTTTTGRAAGYRTTAATGC |
| 5 | AATTCTGGGWAGCGTGGCATTAATACTG |

TABLE 2
Backwards primers, category A

| | |
|---|---|
| No. | Primer sequence |

| | |
|---|---|
| 6 | CCCACTTTAACTGTAAAGGT |
| 7 | CGTCATCATTATATTTTGTATACTCCACC |
| 8 | CACTTGCTGAAAAAAATGAAAG |

TABLE 3
Probes, category A

| | | |
|---|---|---|
| | | Probe |
| No. | Probe sequence | pair |

| | | |
|---|---|---|
| 9 | AGCGTGGCATTAATACTGAATTGTCA | 1 |
| 10 | ATCATGCATCGCGAGTTGCCAGAAT | 1 |
| 11 | GTCCTGCCTGAMTATCATGGACAAGACTCT | 2 |
| 12 | TTCGTGTWGGAAGAATTTCTTTTGRAAGYRTTAAT | 2 |
| 13 | ATGAGTTTCCTTCTATGTGYCCGGYAGATGGAA | 3 |
| 14 | TCCGTGGGATTACGCACAATAAAATATTTGTGGGATT | 3 |
| 15 | AAAYATTATTAATAGCTGCATCRCTTTCATTT | 4 |
| 16 | TTCAGCAAGTGYGCTGGCKRCGCCWGATTCTGTA | 4, 5 |
| 17 | ACTGGRAAGGTGGAGTATACAAAATATAATGAT | 5 |
| 95 | ATTAAYRCTTYCAAAAGAAATTCTTCC | 6 |
| 96 | CAGTATTAATGCCACGCTWCCCAGAATT | 6 |
| 97 | CCTTCTATGTGYCCGGYAGATGGAA | 7 |
| 98 | TSCGTGGGATTACGCACAAT | 7 |

TABLE 4
Forwards primers, category B

| | |
|---|---|
| No. | Primer sequence |

| | |
|---|---|
| 18 | GGCACTGTCTGAAACTGCT |
| 19 | GAAACTGCTCCTGTKTATAC |
| 20 | GATGACRCCGGRAGAMGTG |
| 21 | CTGAACTGGGGGMGAATCAGCAATGTG |

TABLE 5
Backwards primers, category B

| | |
|---|---|
| No. | Primer sequence |

| | |
|---|---|
| 22 | YGCCATTGCATTAACAGA |
| 23 | GCWGCKGTATTACTTTCCCATAA |
| 24 | GGCCTGTCGCCAGTTATCTGACATTCTGGTTG |
| 25 | TCTCTTCATTCACGGCGCG |

TABLE 6
Category C, forwards primers

| | |
|---|---|
| No. | Primer sequence |

| | |
|---|---|
| 26 | GGCGCTGTCTGAGGCATCT |
| 27 | GAGGCATCTCCGCTTTATAC |
| 28 | AATGACGGCTCAGGATGTT |
| 29 | CTGAACTGGGGAAGAATAAGTAATGTT |

Table 7
Category C, backwards primers

| | |
|---|---|
| No. | Primer sequence |

| | |
|---|---|
| 30 | GCAGCGATTGTATTCGCTTCCCACAAAACA |
| 31 | GCCCTGTCTCCAACAATCTGGCATTCTGTTTT |
| 32 | CTGTTTTTGGCTCACGGAACG |
| 33 | CGCCATGGAATTAGCAGAAAAG |

TABLE 8
Probes, category B

| | | |
|---|---|---|
| No. | Probe sequence | Probe pair |

| | | |
|---|---|---|
| 34 | CCCCAGTTCAGWGTGAGGTCC | 1 |
| 35 | CCGGAAGCACATTGCTGATTC | 1 |
| 36 | GAATATCCTTTAATAATATATCAGCGATACTKGG | 2 |
| 37 | WGTGGCSGTTATACTGAATTGYCATCATCAGGG | 2 |
| 38 | CGTTCYGTTCGCKCCGTGAATGAAGAKA | 3 |
| 39 | CAACCAGAATGTCAGATAACTGGCGACAGGCC | 3 |

TABLE 9
Probes, category C

| | | Probe |
|---|---|---|
| No. | Probe sequence | pair |
| 40 | CCCCAGTTCAGGGTAAGGTCA | 1 |
| 41 | CTGGAAGAACATTACTTATTC | 1 |
| 42 | AGGATATCTTTTAATAGTCTTTCTGCGATTCTCGG | 2 |
| 43 | TGTTGCGGTCATCCTTAATTGCCACTCAACCGG | 2 |
| 44 | TTATTCAGTTCGTTCCGTGAGCCAAAAAC | 3 |
| 45 | AAAACAGAATGCCAGATTGTTGGAGACAGGGC | 3 |

TABLE 10
Category D, forwards primers

| | |
|---|---|
| No. | Primer sequence |

| | |
|---|---|
| 46 | CATGCTGCITTTTTAGAAGA |
| 47 | CATGCTGCRTTTTTAGAAGA |
| 48 | CATGCTGCITTTTTAGAAGACTCT |
| 49 | CATGCTGCRTTTTTAGAAGACTCT |
| 50 | AATGAATGGGAAAAGGAGCATGGC |
| 51 | CTCTCTGTCTTTGCTTGCTGATT |
| 52 | CTCGTCAGCATGCAGTAGAAAGAGCAGTCG |
| 53 | CATTGGGATGAGAAGATCGGTGAACTTGCAGG |

TABLE 11
Category D, backwards primers

| | |
|---|---|
| No. | Primer sequence |

| | |
|---|---|
| 54 | CGTCTTTATCTCCGAGYTCAG |
| 55 | ACATCGTCTTTATCTCCGAGYTCAG |
| 56 | TTTACCAACATCCGTCTTATTATAAGATACGG |
| 57 | CCTTCACCAGCAAATACTTCTG |
| 58 | TGAGCCTGCTCCAGAATAAACC |
| 59 | TCAATTTTGAATAATCATATACA |

TABLE 12
Probes, category D

| | | Probe |
|---|---|---|
| No. | Probe sequence | pair |
| 60 | AGAGAAAGAAAACAGAGTGGTAAATATGAATATATGACAT | 1 |
| 61 | TCTTATTGTAAATGGTAAGGATACATGGTCTGTAAAAG | 1 |
| 62 | GGGACCATAGACCTTTCAACAGGTAATGTATCAAGTGTTT | 2 |
| | T | |
| 63 | ACATTTATAACACCAACATTTACCCCAGGAGAAGAAG | 2 |
| 64 | GGCATATATTAATTATCTGGAAAATGGAGGGCTTTTAGAG | 3 |
| | GC | |
| 65 | CAACCGAAGGAGTTTACACAACAAGTGTTTGATCCTC | 3 |
| 66 | CATTGGGATGAGAAGATCGGTGAACTTGCAGGCAT | 4 |
| 67 | AACCCGTAATGCTGATCGCAGTCAGAGTGGTAAGGC | 4 |

TABLE 13
Category E, forwards primers

| | |
|---|---|
| No. | Primer sequence |

| | |
|---|---|
| 68 | GGCCTGGTTACAACATTATGG |
| 69 | ACGCGAAAGATACCGCTCTTGGTAT |
| 70 | CCAGGCTTCGTCACAGTTGCA |
| 71 | GGAACGGCAGAGGTTAATCTGCAG |
| 72 | AGTGGTAATAACTTTGACGGTAGTTC |

TABLE 14
Category E, backwards primers

| | |
|---|---|
| No. | Primer sequence |

| | |
|---|---|
| 73 | ATCCCCATCGTCACCAGA |
| 74 | AACATTATCACCATAATACTG |
| 75 | TAGTTTACACCAACGGTCGCCGC |
| 76 | CATTACCCGTACCATGACGGT |
| 77 | CGGAACTGCATTGAGTAAAGGAGATCA |

TABLE 15
Probes, category E

| | | Probe |
|---|---|---|
| No. | Probe sequence | pair |
| 78 | TCCAGTGAACTACCGTCAAAGTTATYACCAC | 1 |
| 79 | CCAGCATKTTTTCGGAATCATAGAACGGTAATAAGAA | 1 |
| 80 | ATGTTGGGCTATAACGTCTTCATTGATC | 2 |
| 81 | AGGATTTTTCTGGTGATAATACCCGT | 2 |
| 82 | AGGTATTGGTGGCGAATACTGGCGAGACTATTTCAAAAGT | 3 |
| | AG | |
| 83 | TTAACGGCTATTTCCGCATGAGCGGCTGGCATGAGTCAT | 3 |
| | AC | |
| 93 | TCCAGTGAACTACCGTCAAAGTTATYACCAC | 4 |
| 94 | CCAGCATKTTTTCGGAATCATAGAACGGTAATAAGAA | 4 |

In addition to the detection of the EHEC, it is advantageous to control the correct sequence of the method. This invention ensures this control in that it enables the genus-specific detection of E. coli. Especially, a differentiation with respect to enterobacteria, such as the genus citrobacter, is advantageous, because in many cases these bacteria have accepted pathogenicity genes from E. coli in a horizontal gene transfer. An incorrect positive classification as VTEC can therefore be avoided.

Since E. coli and Shigella form one unit from a molecular biological point of view and also in many taxonomical classifications, these two genera are not separated during the control. This is very practicable in practice, because in microbiological routine diagnostics differentiation between these genera does not normally take place.

Tabs. 16+17 contain primers which enable the detection of E. coli and Shigella. For the investigation, aliquots of the same DNA/RNA samples can be used as for the EHEC detection. In addition, it is possible to carry out the E. coli control reaction simultaneously, i.e. in a reaction vessel together with the EHEC detection or in parallel. Furthermore, the E. coli/Shigella detection is also suitable for differentiating these genera from others.

TABLE 16
Category F, forwards primers

| | |
|---|---|
| No. | Primer sequence |

| | |
|---|---|
| 84 | CGG GTC AGG TAA TTG CAC AGT A |
| 85 | CGG GTC AGG TGA TTG CAC AGT A |
| 86 | CGG GTC AGG TGA TTG CAC AAT A |
| 87 | CGG GTC AGG TAA TTG CAC AAT A |

TABLE 17
Category F, backwards primer

| No. | Primer sequence |
|---|---|
| 88 | GCA ACA GTT CAG CAA AGT CCA T |

TABLE 18
Probes, category F

| | | |
|---|---|---|
| No. | Probe sequence | Probe pair |

| | | |
|---|---|---|
| 89 | CGG TGA AGC CAC CGA CAT CGT | 1 |
| 90 | TGG CAG GTT CCG GCC TTC ACT CTC | 1 |
| 91 | AAG CCA CCG ACA TCG TG | 2 |
| 92 | AAG CCA CTG ACA TCG TG | 2 |

The detection of the amplicons can take place through gel electrophoresis and detection of the DNA bands. Alternatively, the amplicons can be detected and quantified with the aid of probes. There are various ways of modifying probes to render a direct or indirect visual indication possible. They can be coupled to an anchor molecule which serves as a linker. This type of anchor molecule may be, for example, a protein which is recognised by an antibody. This antibody may be coupled to an enzyme which causes a colour reaction, whereby the detection is provided. Peroxidase or catalase, for example, are used for these purposes. In addition, a probe can also be radioactively marked, whereby the measurement of the radioactivity leads to the detection and quantification.

Another way is to couple a fluorescent molecule to the probe. In this case it must be ensured that the fluorescence is only emitted or detected when the probe is bound to a single strand of the amplicon. This can be achieved in that the probe-amplicon hybrid is separated from the remaining PCR mixture. For example, probes can be bound to solid surfaces which “trap” the single-strand amplicons, whereby free probes are washed off.

On-line detection of the PCR products presents an elegant method. In this case, a fluorescence signal is only produced when a fluorescence-marked probe settles on an amplicon. This can occur in that the probe part of the amplicon-probe hybrid is selectively enzymatically broken down. Also, due to the opening up of the probe when it binds itself to the amplicon, quenching of the fluorescence signal is cancelled.

A further possibility is that two fluorescence-marked probes are used. It is only when both bind adjacently to an amplicon that a so-called FRET (Fluorescence Resonance Energy Transfer) can produce a signal (FIG. 1). This method has the substantial advantage that several specificity levels are a constituent part of the detection: firstly the primers bind to a certain target molecule, secondly both probes must bind to the “correct” amplicon and thirdly, they must be located adjacently in the correct order. With this adjacent arrangement the distance between the probes is decisive for the successful emission of the signal. Each of these requirements contributes to the increase in the specificity of the detection.

Alternatively, there are also fluorescence molecules which interact with the DNA double helix and then emit a signal. This unspecific detection of PCR products has however the disadvantage that erroneous amplification products are also detected.

According to the above description, the execution of the investigation requires a large number of components. Therefore, it is especially advantageous to offer them in one or more packages of a kit. Such a kit can also contain the reagents and chemicals for enriching the bacteria, the components for the DNA release and purification as well as the consumable material for carrying out the PCR and for the detection.

### DESCRIPTION OF THE FIGURES

FIG. 1 shows the FRET principle schematically.

FIG. 2 shows PCR products with primers of category D.

FIG. 3 shows PCR products with primers of category E.

FIG. 4 shows the amplification and real-time detection of the SltI and SltII genes for EHEC strains.

FIG. 5 shows the amplification and real-time detection of the eae gene for EHEC strains in a multiplex PCR reaction together with the Slt genes.

The following Examples Explain the Invention

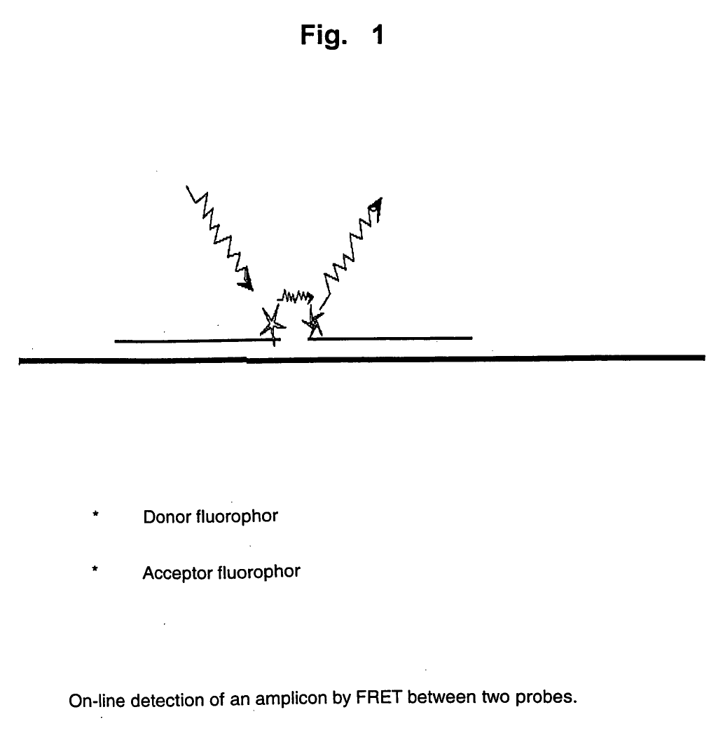

The illustrated FIGS. 1-5 were produced under the following conditions:

FIG. 1: The schematic process of the FRET is shown. Numerous combinations of donor and acceptor are available. However, it is important that the absorption spectrum of the acceptor overlaps with the emission spectrum of the donor. Only then is it ensured that excitation of the donor also leads to an adequately strong fluorescence with the acceptor.

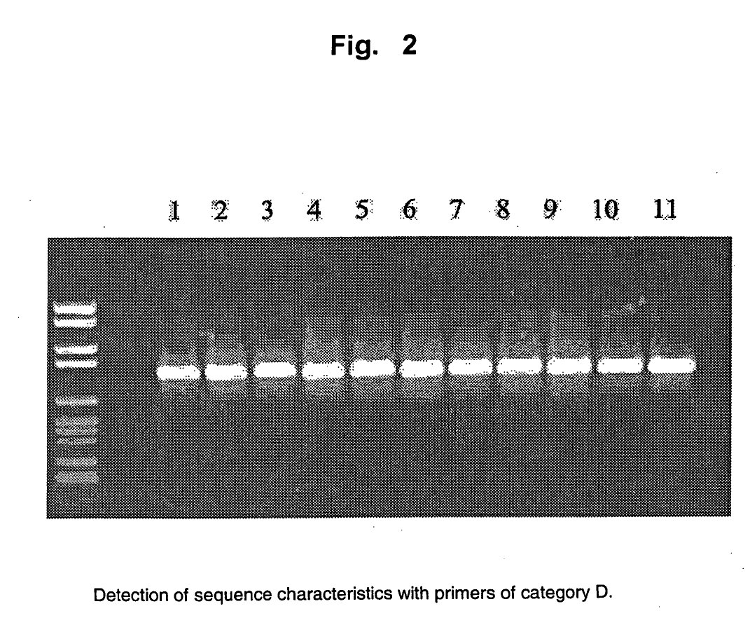

FIG. 2: Detection of EHEC with primers of category D. The test conditions largely correspond to those in the chapter “Detection of EHEC strains by PCR”. The detection in the agarose gel also occurs as described in the above chapter.

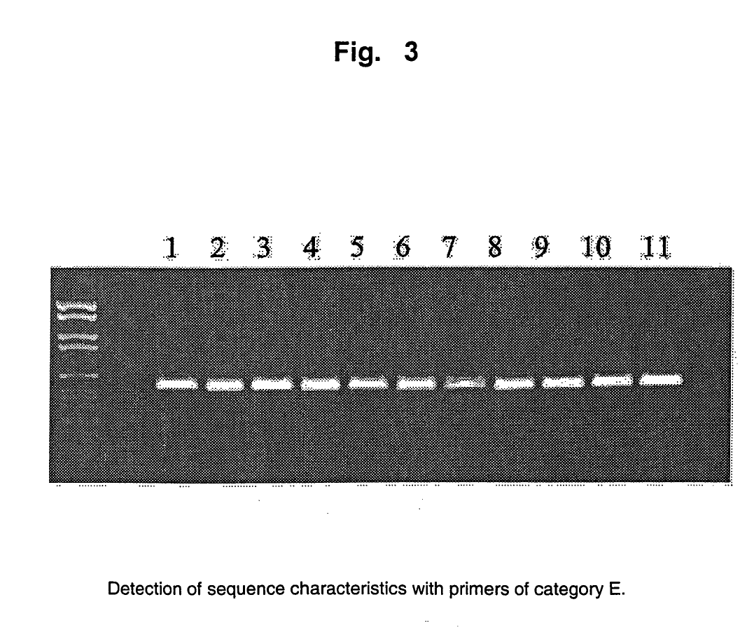

FIG. 3: Detection of EHEC with primers of category E. The test conditions largely correspond to those in the chapter “Detection of EHEC strains by PCR”. The detection in the agarose gel also occurs as described in the above chapter.

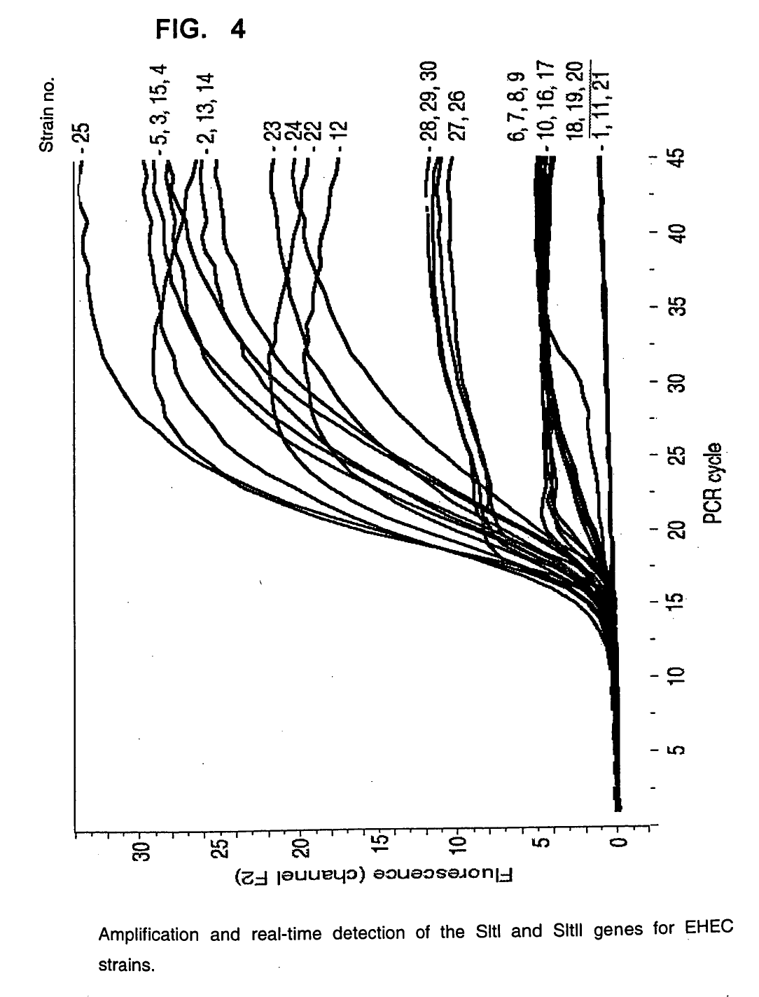

FIG. 4: This shows the amplification of SltI and SltII genes by real-time PCR.

Probes are used which permit the detection of the SltI and also the SltII genes. These were coupled with the same fluorescence colouring agents (Lightcycler RED 640 and Fluorescein) so that the detection only occurs in one channel (F2). It can be seen that with the amplification of the SltII genes, signal curves with amplitudes arise which are larger than 14. The signal curves of the SltI genes lie significantly lower. If SltI and SltII both occur, then the amplitude exhibits the highest level. It is therefore an indicator for the occurrence and the differentiation between SltI and SltII genes.

From FIG. 4 it can also be seen that depending on the application of the various probes, the signal amplitude for the SltI genes is of different heights. For the experiment shown, the primers nos. 1+6 and nos. 18+22 as well as the probes nos. 9+10 (for strain no. 1-10), probes nos. 95+96 (for strain nos. 11-20), probes nos. 97+98 (for strain nos. 21-30) and probes nos. 34+35 (for strains 1-30) were used. The probes were coupled with the colouring agents Fluorescein and Lightcycler Red 640. The detection occurred at a light wavelength of 640 nm.

It can be seen that the probes nos. 97+98 for strains, which only possess the Slt1 gene (see the table on page 56), produce the highest amplitude. This probe-primer combination is therefore especially well suited for on-line PCR.

Detection of the Slt genes: 25 (Slt2 without eae)//5, 15 (Slt2 without eae), 3, 4 (Slt2+eae)//2 (Slt2 without eae) 13, 14 (Slt2+eae)//23 (Slt2+eae)//24 (Slt2+eae)//22 (Slt2 without eae)//12 (Slt2 without eae)//28, 29, 30 (Slt1+eae), 27 (Slt1+eae), 26 (Slt1+Slt2+eae)//6, 16 (Slt1+Slt2+eae), 7, 8, 9, 10, 17, 18, 19, 20 (Slt1+eae)//1, 11, 21 (water).

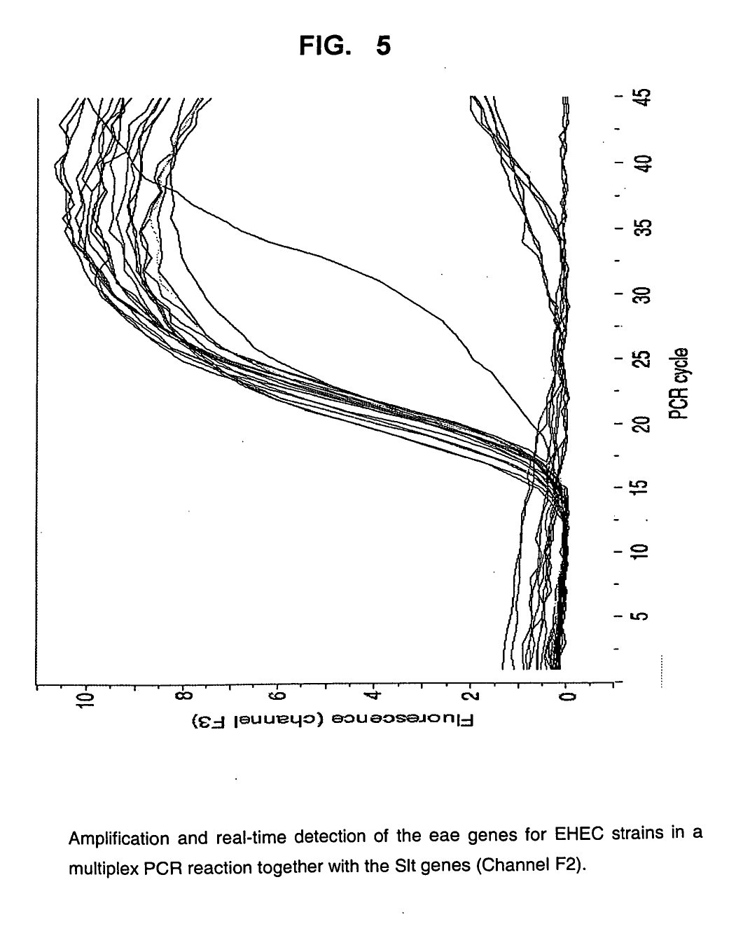

FIG. 5 This shows the amplification and real-time detection of the eae genes for EHEC strains in a multiplex PCR with the Slt genes (FIG. 4).

The multiplex reaction was carried out together with the probes and primers from FIG. 4. For the detection of the eae gene the primers nos. 68+73 and the probes nos. 93+94 were used. The probes nos. 93+94 were coupled with the colouring agents Fluorescein and Lightcycler Red 705. The detection occurred at a light wavelength of 710 nm.

Two groups of curves can be seen. The curves with amplitudes >5 show a positive result for the eae gene. In this respect, strains are involved which possess an eae gene (see legend in FIG. 4, table page 57). The curves with amplitudes <5 indicate a negative result (water samples or strains without the eae gene (samples 1, 11, 21, 5, 15, 25).

Detection of VTEC Strains by PCR

This invention is suitable for the detection of VTEC strains by the polymerase chain reaction. Referred to the complete genome, VTEC strains differ only slightly from conventional E. coli strains. For this reason it is not easy to identify the DNA or RNA sequences which unambiguously map the VTEC group. Since VTEC also exhibits differences within itself, e.g. in the serotypes, a single sequence feature is not suitable for supplying an unambiguous detection.

The invention is based on a combination of several genotypical features being used for the detection, partly simultaneously and where necessary, partly consecutively. In addition primers and probes are provided which exploit the advantages of the PCR for the amplification and detection of the VTEC strains.

Detection of the VTEC strains can occur in various steps, comprising bacterial enrichment, DNA/RNA release and isolation, PCR and (possibly simultaneously) detection of the amplicons.

For enrichment, the bacteria are shaken overnight in 2 ml of LB medium (10 g Bacto Tryptone, 5 g yeast extract, 10 g NaCl in 1 l of water) at 37° C. The bacterial culture was then spun off in a centrifuge at 10000×g and resuspended in 100 μl of water. Then 50 μl 100 mM NaOH were added. The cells were lysated after 5 min. Following this, the solution was neutralised with 100 μl of 0.5 M Tris pH 8. Then the suspension was spun for 10 min. at 10000×g in a centrifuge to remove insoluble constituents. Of this solution 1 μl was used in each case in the PCR reactions.

The PCR reaction was prepared as follows:

Sample volume—1 μl

10×PCR buffer—2.5 μl

10 mM dNTP—0.25 μl

10 μM forwards primer

Category A—0.2 μl

10 μM backwards primer

Category A—0.2 μl

10 μM forwards primer

Category B—0.2 μl

10 μM backwards primer

Category B—0.2 μl

10 μM forwards primer

Category C—0.2 μl

10 μM backwards primer

Category C—0.2 μl

50 mM MgCl₂—0.75 μl

5 U/μl Taq polymerase-0.3 μl

Water—add. 25 μl

The above reaction mixture was firmly closed in 200 μl reaction vessels and incubated according to the following protocol in a PCR device.

95° C.—5 min.

92° C.—1 min.)

52° C.—1 min.×35)

72° C.—0.5 min.)

72° C.—5 min.

In the reaction mixture one forwards and one backwards primer of the categories A, B, C (Tab. 1-9) were used in each case. For example, amplicons for the strains listed in the following table were produced with the primers nos. 1, 6, 18, 22, 26 and 30. Positive results were present for these strains, classified serologically as VTEC, because in each case bands produced by the PCR could be seen in the ethidium-bromide coloured 1% agarose gel.

TABLE
Detection of VTEC strains with the primers of categories A-C

| | Strain no. | | Result |
|---|---|---|---|
| | (Biotecon | VTEC | positive (+)/ |
| | Diagnostics) | serotype | negative (−) |
| 1 | Bc 4734 | O26:H11 | + |
| 2 | Bc 4735 | O157:H- | + |
| 3 | Bc 4736 | | + |
| 4 | Bc 4737 | | + |
| 5 | Bc 4738 | O157:H7 | + |
| 6 | Bc 4945 | O26:H- | + |
| 7 | Bc 4946 | O157:H7 | + |
| 8 | Bc 4947 | O111:H- | + |
| 9 | Bc 4948 | O157:H | + |
| 10 | Bc 4949 | O5 | + |
| 11 | Bc 5643 | O2:H5 | + |
| 12 | Bc 5644 | O128 | + |
| 13 | Bc 5645 | O55:H- | + |
| 14 | Bc 5646 | O69:H- | + |
| 15 | Bc 5647 | O101:H9 | + |
| 16 | Bc 5648 | O103:′H2 | + |
| 17 | Bc 5850 | O22:H8 | + |
| 18 | Bc 5851 | O55:H- | + |
| 19 | Bc 5852 | O48:H21 | + |
| 20 | Bc 5853 | O26:H11 | + |
| 21 | Bc 5854 | O157:H7 | + |
| 22 | Bc 5855 | O157:H- | + |
| 23 | Bc 5856 | O26:H- | + |
| 24 | Bc 5857 | O103:H2 | + |
| 25 | Bc 5858 | O26:H11 | + |
| 26 | Bc 7832 | | + |
| 27 | Bc 7833 | O raw form:H- | + |
| 28 | Bc 7834 | ONT:H- | + |
| 29 | Bc 7835 | O103:H2 | + |
| 30 | Bc 7836 | O57:H- | + |
| 31 | Bc 7837 | ONT:H- | + |
| 32 | Bc 7838 | | + |
| 33 | Bc 7839 | O128:H2 | + |
| 34 | Bc 7840 | O157:H- | + |
| 35 | Bc 7841 | O23:H- | + |
| 36 | Bc 7842 | O157:H- | + |
| 37 | Bc 7843 | | + |
| 38 | Bc 7844 | O157:H- | + |
| 39 | Bc 7845 | O103:H2 | + |
| 40 | Bc 7846 | O26:H11 | + |
| 41 | Bc 7847 | O145:H- | + |
| 42 | Bc 7848 | O157:H- | + |
| 43 | Bc 7849 | O156:H47 | + |
| 44 | Bc 7850 | | + |
| 45 | Bc 7851 | O157:H- | + |
| 46 | Bc 7852 | O157:H- | + |
| 47 | Bc 7853 | O5:H- | + |
| 48 | Bc 7854 | O157:H7 | + |
| 49 | Bc 7855 | O157:H7 | + |
| 50 | Bc 7856 | O26:H- | + |
| 51 | Bc 7857 | | + |
| 52 | Bc 7858 | | + |
| 53 | Bc 7859 | ONT:H- | + |
| 54 | Bc 7860 | O129:H- | + |
| 55 | Bc 7861 | | + |
| 56 | Bc 7862 | O103:H2 | + |
| 57 | Bc 7863 | | + |
| 58 | Bc 7864 | O raw form:H- | + |
| 59 | Bc 7865 | | + |
| 60 | Bc 7866 | O26:H- | + |
| 61 | Bc 7867 | O raw form:H- | + |
| 62 | Bc 7868 | | + |
| 63 | Bc 7869 | ONT:H- | + |
| 64 | Bc 787O | O113:H- | + |
| 65 | Bc 7871 | ONT:H- | + |
| 66 | Bc 7872 | ONT:H- | + |
| 67 | Bc 7873 | | + |
| 68 | Bc 7874 | O raw form:H- | + |
| 69 | Bc 7875 | O157:H- | + |
| 70 | Bc 7876 | O111:H- | + |
| 71 | Bc 7877 | O146:H21 | + |
| 72 | Bc 7878 | O145:H- | + |
| 73 | Bc 7879 | O22:H8 | + |
| 74 | Bc 7880 | O raw form:H- | + |
| 75 | Bc 7881 | O145:H- | + |
| 76 | Bc 8275 | O157:H7 | + |
| 77 | Bc 8318 | O55:K-:H- | + |
| 78 | Bc 8325 | O157:H7 | + |
| 79 | Bc 8333 | | + |
| 80 | Bc 8332 | ONT | + |
| 81 | Bc 5580 | O157:H7 | + |
| 82 | Bc 5582 | O3:H | + |
| 83 | Bc 5579 | O157:H7 | + |

In addition the amplicons could be detected with fluorescence-marked probe pairs from the categories A, B and C, that is, for example, with the probes SEQ ID no. 9, 10, 34, 35, 95, 96, 97, 98 and 40+41.

Detection of EHEC Strains by PCR

Enterohemorrhagic E. coli can cause severe diarrhoea illnesses as germs contaminating foodstuffs. They are responsible for the HUS (haemolytic-uraemic syndrome), characterised by blood-containing diarrhoea and acute kidney failure. The illness can be fatal.

The EHEC can systematically be regarded as a subgroup of the VTEC. For this reason the detection can occur in two stages in which firstly the VTEC are detected according to Example 1 and then the EHEC detection occurs from the positive findings.

In this example strains in the following table are examined:

| No. | Biotecon No. | Sero var. | VTEC +/− | EHEC +/− |
|---|---|---|---|---|
| 1 | BC 12503 | O157H- | + | + |
| 2 | BC 12507 | O157H- | + | + |
| 3 | BC 12408 | O84H21 | + | + |
| 4 | BC 12518 | O157H7 | + | + |
| 5 | BC 12530 | O156H- | + | + |
| 6 | BC 12538 | O157H7 | + | + |
| 7 | BC 12543 | O111H- | + | + |
| 8 | BC 12544 | O26H11 | + | + |
| 9 | BC 12545 | O103H2 | + | + |
| 10 | BC 12546 | O118H- | + | + |
| 11 | BC 12547 | O118H- | + | + |

The detection of the EHEC strains can occur in various steps, comprising bacterial enrichment, DNA/RNA release and isolation, PCR and (possibly simultaneously) detection of the amplicons.

For enrichment the bacteria are shaken overnight in 2 ml LB medium (10 g Bacto Tryptone, 5 g yeast extract, 10 g NaCl in 1 l of water) at 37° C. The bacterial culture was then spun off in a centrifuge at 10000×g and resuspended in 100 μl of water. Then 50 μl 100 mM NaOH were added. The cells were lysated after 5 min. Following this, the solution was neutralised with 100 μl of 0.5 M Tris pH 8. Then the suspension was spun for 10 min. at 10000×g in a centrifuge to remove insoluble constituents. Of this solution 1 μl was used in each case in the PCR reactions.

The PCR reaction was prepared as follows:

Sample volume—1 μl

10×PCR buffer—2.5 μl

10 mM dNTP—0.25 μl

10 μM forwards primer

Category A—0.2 μl

10 μM backwards primer

Category A—0.2 μl

10 μM forwards primer

Category B—0.2 μl

10 μM backwards primer

Category B—0.2 μl

10 μM forwards primer

Category C—0.2 μl

10 μM backwards primer

Category C—0.2 μl

50 mM MgCl₂—0.75 μl

5 U/μl Taq polymerase—0.3 μl

Water—add. 25 μl

The above reaction mixture was firmly closed in 200 μl reaction vessels and incubated according to the following protocol in a PCR device.

95° C.—5 min.

92° C.—1 min.)

52° C.—1 min.×35)

72° C.—0.5 min.)

72° C.—5 min.

In the reaction mixture one forwards and one backwards primer of the categories A, B, C (Tab. 1-9) were used in each case. For example, amplicons were produced with the primers nos. 1, 6, 18, 22, 26 and 30. Positive results were present for these strains, classified serologically as EHEC, because in each case bands produced by the PCR could be seen in the ethidium-bromide coloured 1% agarose gel.

The DNA of the positive results was again examined in a second run. In this run a PCR with forwards and backwards primers of the categories D and E is used. The following protocol is used:

Sample volume—1 μl

10×PCR buffer—2.5 μl

10 mM dNTP—0.25 μl

10 μM forwards primer

Category C—0.2 μl

10 μM backwards primer

Category C—0.2 μl

10 μM forwards primer

Category D—0.2 μl

10 μM backwards primer

Category D—0.2 μl

50 mM MgCl₂—0.75 μl

5 U/μl Taq polymerase—0.3 μl

Water—add. 25 μl

The above reaction mixture was firmly closed in 200 μl reaction vessels and incubated according to the following protocol in a PCR device.

95° C.—5 min.

92° C.—1 min.)

52° C.—1 min.×35)

72° C.—0.5 min.)

72° C.—5 min.

As a primer of category D, for example, the combination of primers nos. 46, 54 and nos. 68 and 73 can be used. It is also possible to use this primer pair in parallel PCR reactions. The results from two separate PCR runs are illustrated in the following.

Since the bands of FIGS. 2 and 3 have different sizes, they can also be detected in a gel, originating from a single PCR reaction, as double bands. Furthermore, the bands can be detected by the previously described FRET technology in that probe pairs of categories D and E are used. For example, the probes nos. 60, 61 and 78, 79 can be used for this purpose.

Specificity of the EHEC Detection

As previously described, the EHEC detection preferably occurs in at least two steps, comprising PCR reactions with the primer categories A-C and D-E. Here, positive results from the first step are further examined in a second step. If the first step turns out to be negative, this result can be checked by an appropriate control in which E. coli is detected. Furthermore, it is important that the primers of categories A-C do not indicate any incorrect positive results. For this reason their specificity has been intensively examined. The results are presented in the following.

For enrichment the bacteria are shaken overnight in 2 ml LB medium (10 g Bacto Tryptone, 5 g yeast extract, 10 g NaCl in 1 l of water) at 37° C. The bacterial culture was then spun off in a centrifuge at 10000×g and resuspended in 100 μl of water. Then 50 μl 100 mM NaOH were added. The cells were lysated after 5 min. Following this, the solution was neutralised with 100 μl of 0.5 M Tris pH 8. The suspension was then spun for 10 min. at 10000×g in a centrifuge to remove insoluble constituents. Of this solution 1 μl was used in each case in the PCR reactions.

The PCR reaction was prepared as follows:

Sample volume—1 μl

10×PCR buffer—2.5 μl

10 mM dNTP—0.25 μl

10 μM forwards primer

Category A—0.2 μl

10 μM backwards primer

Category A—0.2 μl

10 μM forwards primer

Category B—0.2 μl

10 μM backwards primer

Category B—0.2 μl

10 μM forwards primer

Category C—0.2 μl

10 μM backwards primer

Category C—0.2 μl

50 mM MgCl₂—0.75 μl

5 U/μl Taq polymerase—0.3 μl

Water—add. 25 μl

The above reaction mixture was firmly closed in 200 μl reaction vessels and incubated according to the following protocol in a PCR device.

95° C.—5 min.

92° C.—1 min.)

52° C.—1 min.×35)

72° C.—0.5 min.)

72° C.—5 min.

One forwards and one backwards primer of the categories A, B, C (Tab. 1-9) in each case was used in the reaction mixture. For example, with the primers nos. 1, 6, 18, 22, 26 and 30 no amplicons were produced with the strains listed in the following table. Negative results were consequently present for these strains, because in no case could bands of the expected size produced by the PCR be seen in the ethidium-bromide coloured 1% agarose gel. Since the correct DNA fragments were not amplified, also no incorrect positive result can arise due to probes of the categories A-C. This, too, was experimentally verified.

TABLE
Bacterial strains tested as negative controls

| Species | Strain no. | PCR detection |
|---|---|---|
| Aeromonas hydrophila | DSM 30188 | − |
| Pseudomonas cepacia | BC 3134 | − |
| Pseudomonas paucimobilis | DSM 1098 | − |
| Lactobacillus bifermentans | BC 8463 | − |
| Flavobacterium johnsonii | DSM 2064 | − |
| Flavobacterium flavense | DSM 1076 | − |
| Flavobacterium resinovorum | DSM 7478 | − |
| Enterococcus casseliflavus | BC 7629 | − |
| Comamonas testosteroni | BC 4276 | − |
| Alcaligenes latus | DSM 1122 | − |
| Budvicia aquatica | BC 8923 | − |
| Achromobacter ruhlandii | BC 8908 | − |
| Achromobacter xylosa | BC 8913 | − |
| Sphingobacterium multivorans | BC 8924 | − |
| Ralstonia pickettii | BC 5368 | − |
| Sphingomonas paucimobilis | BC 5293 | − |
| Acinetobacter calcoaceticus | DSM 590 | − |
| Aeromonas hydrophila | DSM 6173 | − |
| Aeromonas enteropeloges | DSM 6394 | − |
| Moraxella catarrhalis | DSM 9143 | − |
| Pasteurella pneumotropica | DSM 2891 | − |
| Pseudomonas beijerinkii | DSM 7218 | − |
| Stenotrophomonas putrefaciens | BC 5337 | − |
| Xanthomonas maltophila | BC 4273 | − |
| Brochotrix thermosphacta | DSM 20171 | − |
| Brochotrix thermophilus | DSM 20594 | − |
| Brochotrix campestris | DSM 4712 | − |
| Staphylococcus haemolyticus | BC 2747 | − |
| Staphylococcus | BC 5468 | − |
| chromogenes | | |
| Staphylococcus gallinosum | BC 5472 | − |
| Staphylococcus lentus | BC 5462 | − |
| Staphylococcus intermedius | DSM 20036 | − |
| Staphylococcus | DSM 20038 | − |
| saprophyticus | | |
| Staphylococcus hominis | BC 5466 | − |
| Staphylococcus equorum | BC 9447 | − |
| Staphylococcus sciuri | BC 5461 | − |
| Staphylococcus hyicus | BC 5469 | − |
| Aeromonas caviae | DSM 7326 | − |
| Pantoea stewartii | DSM 30176 | − |
| Xhenorhabdus poinarii | DSM 4768 | − |
| Klebsiella ornitholytica | DSM 7464 | − |
| Vibrio vulnificus | DSM 10147 | − |
| Moellerella wisconsis | DSM 5079 | − |
| Yersinia pseudotuberculosis | BC 8723 | − |
| Vibrio mimicus | DSM 33653 | − |
| Aeromonas sobriae | ATCC 43979 | − |
| Pasteurella aerogenes | DSM 10153 | − |
| Listonella anguillarum | DSM 11323 | − |

Use of E. coli Positive Controls

As described previously, EHEC strains are detected according to the invention in two steps by using the primers A-C and D-E. If the PCR reactions of the first step indicate a positive result, the samples are examined further in a second step. If on the other hand Step 1 turns out to be negative, then there is no VTEC and therefore also no EHEC strain present. However, it must be ensured that experimental errors can be eliminated. One possibility involves the detection of E. coli, because this germ is present in almost all foodstuffs relevant to EHEC. By doping a foodstuff with an E. coli strain there is the possibility of using this harmless control germ on a routine basis. In addition detection of E. coli is often desired from a hygiene point of view.

From pure cultures of the bacteria listed in the following table genomic DNA was isolated using a familiar standard method. Approximately 1 to 10 ng of each of these preparations were then used in the presence of each of 0.4 μM of an equimolar oligonucleotide mixture nos. 84-87 and 0.4 μM oligonucleotide no. 88, 2 mM MgCl₂, 200 μM dNTP (Roche Diagnostics, dUTP was used instead of dTTP), and 0.03 U/μl Taq polymerase (Life Technologies) in a single concentrated reaction buffer (Life Technologies) in the PCR. The PCR was carried out in a Perkin Elmer 9600 Thermocycler with the following listed thermal profile:

| | | |
|---|---|---|
| Initial denaturing | 95° C. | 5 min. |
| Amplification (35 cycles) | 95° C. | 20 s. |
| | 63° C. | 45 s. |
| Final synthesis | 72° C. | 5 min. |

After termination of the PCR reaction the amplification products were fractionated using agarose-gel electrophoresis and rendered visible by colouration with ethidium bromide. The expected products of a length of 351 base pairs where only observed in the cases in which DNA of strains of the species E. coli or the genus Shigella was present. The DNA fractionated in the gels was transferred to nylon filters in a familiar standard method and hybridised for checking the specificity with the oligonucleotides nos. 91 and 92 marked on the 5′ end with biotin. The hybridisation occurred in 5×SSC, 2% blocking reagent, 0.1% lauroyl sarcosine, 0.02% SDS and 5 pmol/ml of probe for 4 hrs at 52° C. Washing took place in 2×SSC, 0.1% SDS for 2×10 min. at 52° C. The detection occurred in a familiar standard method using alkaline phosphatase conjugates (ExtrAvidin, Sigma) in the presence of 5-bromo-4-chloro-3-indolyl phosphate and 4-nitro blue tetrazolium chloride (Boehringer Mannheim). On the filters a band was observed only in those cases in which a band of 351 base pairs were previously visible on the agarose gel. Hence, the presence of all 645 tested E. coli and 32 Shigella strains was detected (see following table) using PCR and hybridisation. In contrast, none of the tested bacterial strains not belonging to this species was acquired with this system.

TABLE
List of the tested bacteria of the E. coli/Shigella group

| | | | | PCR | Hybridisation |
|---|---|---|---|---|---|
| Species | Strain no. | Serotype | Pathotype | detection | with probes |
| E. coli | NCTC 12757 | n.d. | | + | + |
| E. coli | NCTC 12779 | n.d. | | + | + |
| E. coli | NCTC 12790 | n.d. | | + | + |
| E. coli | NCTC 12796 | n.d. | | + | + |
| E. coli | NCTC 12811 | n.d. | | + | + |
| E. coli | ATCC 11229 | n.d. | | + | + |
| E. coli | ATCC 25922 | n.d. | | + | + |
| E. coli | ATCC 8739 | n.d. | | + | + |
| E. coli | DSM 30083 | O1:K1:H7 | | + | + |
| E. coli | BC 5849 | O111:H2 | | + | + |
| E. coli | BC 8265 | O104 | | + | + |
| E. coli | BC 8267 | O55 | | + | + |
| E. coli | BC 8268 | O6:H16 | | + | + |
| E. coli | BC 8270 | O55:K(59):H- | | + | + |
| E. coli | BC 8271 | O55 | | + | + |
| E. coli | BC 8272 | O55:K-:H- | | + | + |
| E. coli | BC 8273 | O55 | | + | + |
| E. coli | BC 8276 | O128:K-H- | | + | + |
| E. coli | BC 8277 | O128:K68:H2 | | + | + |
| E. coli | BC 8278 | O126 | | + | + |
| E. coli | BC 8279 | O126 | | + | + |
| E. coli | BC 8312 | ONT:H- | | + | + |
| E. coli | BC 8317 | O158:K-:H23 | | + | + |
| E. coli | BC 8319 | O128:H21 | | + | + |
| E. coli | BC 8320 | O55:H- | | + | + |
| E. coli | BC 8321 | O55 | | + | + |
| E. coli | BC 8322 | O55 | | + | + |
| E. coli | BC 8326 | O104 | | + | + |
| E. coli | BC 8327 | 037 | | + | + |
| E. coli | BC 8331 | O24 | | + | + |
| E. coli | BC 8335 | O119:H27 | | + | + |
| E. coli | BC 8338 | O10:H4 | | + | + |
| E. coli | BC 8341 | O110:H17 | | + | + |
| E. coli | BC 8344 | O103 | | + | + |
| E. coli | BC 8345 | O103 | | + | + |
| E. coli | BC 8346 | O44 | | + | + |
| E. coli | BC 8347 | O44 | | + | + |
| E. coli | BC 8348 | O44 | | + | + |
| E. coli | BC 8863 | n.d. | | + | + |
| E. coli | BC 8864 | n.d. | | + | + |
| E. coli | BC 4734 | O26:H11 | VTEC | + | + |
| E. coli | BC 4735 | O157:H- | VTEC | + | + |
| E. coli | BC 4736 | n.d. | VTEC | + | + |
| E. coli | BC 4737 | n.d. | VTEC | + | + |
| E. coli | BC 4738 | O157:H7 | VTEC | + | + |
| E. coli | BC 4945 | O26:H- | VTEC | + | + |
| E. coli | BC 4946 | O157:H7 | VTEC | + | + |
| E. coli | BC 4947 | O111:H- | VTEC | + | + |
| E. coli | BC 4948 | O157:H | VTEC | + | + |
| E. coli | BC 4949 | O5 | VTEC | + | + |
| E. coli | BC 5579 | O157:H7 | VTEC | + | + |
| E. coli | BC 5580 | O157:H7 | VTEC | + | + |
| E. coli | BC 5582 | O3:H | VTEC | + | + |
| E. coli | BC 5643 | O2:H5 | VTEC | + | + |
| E. coli | BC 5644 | O128 | VTEC | + | + |
| E. coli | BC 5645 | O55:H- | VTEC | + | + |
| E. coli | BC 5646 | O69:H- | VTEC | + | + |
| E. coli | BC 5647 | O101:H9 | VTEC | + | + |
| E. coli | BC 5648 | O103:H2 | VTEC | + | + |
| E. coli | BC 5850 | O22:H8 | VTEC | + | + |
| E. coli | BC 5851 | O55:H- | VTEC | + | + |
| E. coli | BC 5852 | O48:H21 | VTEC | + | + |
| E. coli | BC 5853 | O26:H11 | VTEC | + | + |
| E. coli | BC 5854 | O157:H7 | VTEC | + | + |
| E. coli | BC 5855 | O157:H- | VTEC | + | + |
| E. coli | BC 5856 | O26:H- | VTEC | + | + |
| E. coli | BC 5857 | O103:H2 | VTEC | + | + |
| E. coli | BC 5858 | O26:H11 | VTEC | + | + |
| E. coli | BC 7832 | n.d. | VTEC | + | + |
| E. coli | BC 7833 | O raw form:H- | VTEC | + | + |
| E. coli | BC 7834 | ONT:H- | VTEC | + | + |
| E. coli | BC 7835 | O103:H2 | VTEC | + | + |
| E. coli | BC 7836 | O57:H- | VTEC | + | + |
| E. coli | BC 7837 | ONT:H- | VTEC | + | + |
| E. coli | BC 7838 | n.d. | VTEC | + | + |
| E. coli | BC 7839 | O128:H2 | VTEC | + | + |
| E. coli | BC 7840 | O157:H- | VTEC | + | + |
| E. coli | BC 7841 | O23:H- | VTEC | + | + |
| E. coli | BC 7842 | O157:H- | VTEC | + | + |
| E. coli | BC 7843 | n.d. | VTEC | + | + |
| E. coli | BC 7844 | O157:H- | VTEC | + | + |
| E. coli | BC 7845 | O103:H2 | VTEC | + | + |
| E. coli | BC 7846 | O26:H11 | VTEC | + | + |
| E. coli | BC 7847 | O145:H- | VTEC | + | + |
| E. coli | BC 7848 | O157:H- | VTEC | + | + |
| E. coli | BC 7849 | O156:H47 | VTEC | + | + |
| E. coli | BC 7850 | n.d. | VTEC | + | + |
| E. coli | BC 7851 | O157:H- | VTEC | + | + |
| E. coli | BC 7852 | O157:H- | VTEC | + | + |
| E. coli | BC 7853 | O5:H- | VTEC | + | + |
| E. coli | BC 7854 | O157:H7 | VTEC | + | + |
| E. coli | BC 7855 | O157:H7 | VTEC | + | + |
| E. coli | BC 7856 | O26:H- | VTEC | + | + |
| E. coli | BC 7857 | n.d. | VTEC | + | + |
| E. coli | BC 7858 | n.d. | VTEC | + | + |
| E. coli | BC 7859 | ONT:H- | VTEC | + | + |
| E. coli | BC 7860 | O129:H- | VTEC | + | + |
| E. coli | BC 7861 | n.d. | VTEC | + | + |
| E. coli | BC 7862 | O103:H2 | VTEC | + | + |
| E. coli | BC 7863 | n.d. | VTEC | + | + |
| E. coli | BC 7864 | O raw form:H- | VTEC | + | + |
| E. coli | BC 7865 | n.d. | VTEC | + | + |
| E. coli | BC 7866 | O26:H- | VTEC | + | + |
| E. coli | BC 7867 | O raw form:H- | VTEC | + | + |
| E. coli | BC 7868 | n.d. | VTEC | + | + |
| E. coli | BC 7869 | ONT:H- | VTEC | + | + |
| E. coli | BC 7870 | O113:H- | VTEC | + | + |
| E. coli | BC 7871 | ONT:H- | VTEC | + | + |
| E. coli | BC 7872 | ONT:H- | VTEC | + | + |
| E. coli | BC 7873 | n.d. | VTEC | + | + |
| E. coli | BC 7874 | O raw form:H- | VTEC | + | + |
| E. coli | BC 7875 | O157:H- | VTEC | + | + |
| E. coli | BC 7876 | O111:H- | VTEC | + | + |
| E. coli | BC 7877 | O146:H21 | VTEC | + | + |
| E. coli | BC 7878 | O145:H- | VTEC | + | + |
| E. coli | BC 7879 | O22:H8 | VTEC | + | + |
| E. coli | BC 7880 | O raw form:H- | VTEC | + | + |
| E. coli | BC 7881 | O145:H- | VTEC | + | + |
| E. coli | BC 8275 | O157:H7 | VTEC | + | + |
| E. coli | BC 8318 | O55:K-:H- | VTEC | + | + |
| E. coli | BC 8325 | O157:H7 | VTEC | + | + |
| E. coli | BC 8332 | ONT | VTEC | + | + |
| E. coli | BC 8333 | n.d. | VTEC | + | + |
| E. coli | BC 8246 | O152:K-:H- | EIEC | + | + |
| E. coli | BC 8247 | O124:K(72):H3 | EIEC | + | + |
| E. coli | BC 8248 | O124 | EIEC | + | + |
| E. coli | BC 8249 | O112 | EIEC | + | + |
| E. coli | BC 8250 | O136:K(78):H- | EIEC | + | + |
| E. coli | BC 8251 | O124:H- | EIEC | + | + |
| E. coli | BC 8252 | O144:K-:H- | EIEC | + | + |
| E. coli | BC 8253 | O143:K:H- | EIEC | + | + |
| E. coli | BC 8254 | O143 | EIEC | + | + |
| E. coli | BC 8255 | O112 | EIEC | + | + |
| E. coli | BC 8256 | O28a.e | EIEC | + | + |
| E. coli | BC 8257 | O124:H- | EIEC | + | + |
| E. coli | BC 8258 | O143 | EIEC | + | + |
| E. coli | BC 8259 | O167:K-:H5 | EIEC | + | + |
| E. coli | BC 8260 | O128a.c.:H35 | EIEC | + | + |
| E. coli | BC 8261 | O164 | EIEC | + | + |
| E. coli | BC 8262 | O164:K-:H- | EIEC | + | + |
| E. coli | BC 8263 | O164 | EIEC | + | + |
| E. coli | BC 8264 | O124 | EIEC | + | + |
| E. coli | BC 7567 | O86 | EPEC | + | + |
| E. coli | BC 7568 | O128 | EPEC | + | + |
| E. coli | BC 7571 | O114 | EPEC | + | + |
| E. coli | BC 7572 | O119 | EPEC | + | + |
| E. coli | BC 7573 | O125 | EPEC | + | + |
| E. coli | BC 7574 | O124 | EPEC | + | + |
| E. coli | BC 7576 | O127a | EPEC | + | + |
| E. coli | BC 7577 | O126 | EPEC | + | + |
| E. coli | BC 7578 | O142 | EPEC | + | + |
| E. coli | BC 7579 | O26 | EPEC | + | + |
| E. coli | BC 7580 | OK26 | EPEC | + | + |
| E. coli | BC 7581 | O142 | EPEC | + | + |
| E. coli | BC 7582 | O55 | EPEC | + | + |
| E. coli | BC 7583 | O158 | EPEC | + | + |
| E. coli | BC 7584 | O- | EPEC | + | + |
| E. coli | BC 7585 | O- | EPEC | + | + |
| E. coli | BC 7586 | O- | EPEC | + | + |
| E. coli | BC 8330 | n.d. | EPEC | + | + |
| E. coli | BC 8550 | O26 | EPEC | + | + |
| E. coli | BC 8551 | O55 | EPEC | + | + |
| E. coli | BC 8552 | O158 | EPEC | + | + |
| E. coli | BC 8553 | O26 | EPEC | + | + |
| E. coli | BC 8554 | O158 | EPEC | + | + |
| E. coli | BC 8555 | O86 | EPEC | + | + |
| E. coli | BC 8556 | O128 | EPEC | + | + |
| E. coli | BC 8557 | OK26 | EPEC | + | + |
| E. coli | BC 8558 | O55 | EPEC | + | + |
| E. coli | BC 8560 | O158 | EPEC | + | + |
| E. coli | BC 8561 | O158 | EPEC | + | + |
| E. coli | BC 8562 | O114 | EPEC | + | + |
| E. coli | BC 8563 | O86 | EPEC | + | + |
| E. coli | BC 8564 | O128 | EPEC | + | + |
| E. coli | BC 8565 | O158 | EPEC | + | + |
| E. coli | BC 8566 | O158 | EPEC | + | + |
| E. coli | BC 8567 | O158 | EPEC | + | + |
| E. coli | BC 8568 | O111 | EPEC | + | + |
| E. coli | BC 8569 | O128 | EPEC | + | + |
| E. coli | BC 8570 | O114 | EPEC | + | + |
| E. coli | BC 8571 | O128 | EPEC | + | + |
| E. coli | BC 8572 | O128 | EPEC | + | + |
| E. coli | BC 8573 | O158 | EPEC | + | + |
| E. coli | BC 8574 | O158 | EPEC | + | + |
| E. coli | BC 8575 | O158 | EPEC | + | + |
| E. coli | BC 8576 | O158 | EPEC | + | + |
| E. coli | BC 8577 | O158 | EPEC | + | + |
| E. coli | BC 8578 | O158 | EPEC | + | + |
| E. coli | BC 8581 | O158 | EPEC | + | + |
| E. coli | BC 8583 | O128 | EPEC | + | + |
| E. coli | BC 8584 | O158 | EPEC | + | + |
| E. coli | BC 8585 | O128 | EPEC | + | + |
| E. coli | BC 8586 | O158 | EPEC | + | + |
| E. coli | BC 8588 | O26 | EPEC | + | + |
| E. coli | BC 8589 | O86 | EPEC | + | + |
| E. coli | BC 8590 | O127 | EPEC | + | + |
| E. coli | BC 8591 | O128 | EPEC | + | + |
| E. coli | BC 8592 | O114 | EPEC | + | + |
| E. coli | BC 8593 | O114 | EPEC | + | + |
| E. coli | BC 8594 | O114 | EPEC | + | + |
| E. coli | BC 8595 | O125 | EPEC | + | + |
| E. coli | BC 8596 | O158 | EPEC | + | + |
| E. coli | BC 8597 | O26 | EPEC | + | + |
| E. coli | BC 8598 | O26 | EPEC | + | + |
| E. coli | BC 8599 | O158 | EPEC | + | + |
| E. coli | BC 8605 | O158 | EPEC | + | + |
| E. coli | BC 8606 | O158 | EPEC | + | + |
| E. coli | BC 8607 | O158 | EPEC | + | + |
| E. coli | BC 8608 | O128 | EPEC | + | + |
| E. coli | BC 8609 | O55 | EPEC | + | + |
| E. coli | BC 8610 | O114 | EPEC | (+) | + |
| E. coli | BC 8615 | O158 | EPEC | + | + |
| E. coli | BC 8616 | O128 | EPEC | + | + |
| E. coli | BC 8617 | O26 | EPEC | + | + |
| E. coli | BC 8618 | O86 | EPEC | + | + |
| E. coli | BC 8619 | n.d. | EPEC | + | + |
| E. coli | BC 8620 | n.d. | EPEC | + | + |
| E. coli | BC 8621 | n.d. | EPEC | + | + |
| E. coli | BC 8622 | n.d. | EPEC | + | + |
| E. coli | BC 8623 | n.d. | EPEC | + | + |
| E. coli | BC 8624 | O158 | EPEC | + | + |
| E. coli | BC 8625 | O158 | EPEC | + | + |
| E. coli | BC 5581 | O78:H11 | ETEC | + | + |
| E. coli | BC 5583 | O2:K1 | ETEC | + | + |
| E. coli | BC 8221 | O118 | ETEC | + | + |
| E. coli | BC 8222 | O148:H- | ETEC | + | + |
| E. coli | BC 8223 | O111 | ETEC | + | + |
| E. coli | BC 8224 | O110:H- | ETEC | + | + |
| E. coli | BC 8225 | O148 | ETEC | + | + |
| E. coli | BC 8226 | O118 | ETEC | + | + |
| E. coli | BC 8227 | O25:H42 | ETEC | + | + |
| E. coli | BC 8229 | O6 | ETEC | + | + |
| E. coli | BC 8231 | O153:H45 | ETEC | + | + |
| E. coli | BC 8232 | O9 | ETEC | + | + |
| E. coli | BC 8233 | O148 | ETEC | + | + |
| E. coli | BC 8234 | O128 | ETEC | + | + |
| E. coli | BC 8235 | O118 | ETEC | + | + |
| E. coli | BC 8237 | O111 | ETEC | + | + |
| E. coli | BC 8238 | O110:H17 | ETEC | + | + |
| E. coli | BC 8240 | O148 | ETEC | + | + |
| E. coli | BC 8241 | O6H16 | ETEC | + | + |
| E. coli | BC 8243 | O153 | ETEC | + | + |
| E. coli | BC 8244 | O15:H- | ETEC | + | + |
| E. coli | BC 8245 | O20 | ETEC | + | + |
| E. coli | BC 8269 | O125a.c:H- | ETEC | + | + |
| E. coli | BC 8313 | O6:H6 | ETEC | + | + |
| E. coli | BC 8315 | O153:H- | ETEC | + | + |
| E. coli | BC 8329 | n.d. | ETEC | + | + |
| E. coli | BC 8334 | O118:H12 | ETEC | + | + |
| E. coli | BC 8339 | n.d. | ETEC | + | + |
| E. coli clinical isolates | | | | 359 (359) | 359 (359) |
| E. coli food isolates | | | | 12 (12) | 12 (12) |
| E. coli environmental isolates | | | | 23 (23) | 23 (23) |
| Shigella boydii | DSM 7532 | 2 | | + | + |
| Sh. boydii | BC 7545 | 1 | | + | + |
| Sh. boydii | BC 7546 | 2 | | + | + |
| Sh. boydii | BC 7547 | 3 | | + | + |
| Sh. boydii | BC 7548 | 4 | | + | + |
| Sh. boydii | BC 7549 | 5 | | + | + |
| Sh. boydii | BC 7550 | 6 | | + | + |
| Sh. boydii | BC 7551 | 7 | | + | + |
| Sh. boydii | BC 7552 | 8 | | + | + |
| Sh. dysenteriae | NCTC 4837 | 1 | | + | + |
| Sh. dysenteriae | BC 7566 | 1 | | + | + |
| Sh. dysenteriae | BC 7553 | 2 | | + | + |
| Sh. dysenteriae | BC 7554 | 3 | | + | + |
| Sh. dysenteriae | BC 7555 | 5 | | + | + |
| Sh. dysenteriae | BC 7556 | 7 | | + | + |
| Sh. dysenteriae | BC 7557 | 8 | | + | + |
| Sh. dysenteriae | BC 7559 | 10 | | + | + |
| Sh. flexneri | DSM 4782 | 2a | | + | + |
| Sh. flexneri | BC 5935 | 1a | | + | + |
| Sh. flexneri | BC 5936 | 2a | | + | + |
| Sh. flexneri | BC 5937 | 6 | | + | + |
| Sh. flexneri | BC 7560 | 1b | | + | + |
| Sh. flexneri | BC 7561 | 2a | | + | + |
| Sh. flexneri | BC 7562 | 3b | | + | + |
| Sh. flexneri | BC 7563 | 4 | | + | + |
| Sh. flexneri | BC 7564 | 5 | | + | + |
| Sh. flexneri | BC 7565 | 6 | | + | + |
| Shigella sonnei | BC 1201 | | | + | + |
| Shigella sonnei | BC 4302 | | | + | + |
| Shigelle sonnei | BC 4301 | | | + | + |
| Shigella sonnei | BC 7889 | | | + | + |
| Shigella sp. | BC 4303 | | | + | + |

ATCC: American Type Culture Collection (Manassas, USA)
BC: Strain Collection at BioteCon GmbH
DSM: German Collection of Micro-organisms (Braunschweig, Germany)
NCTC: National Collection of Type Cultures (London, United Kingdom)
+ = positive reaction
− = negative reaction
(+) = weak positive reaction
n.d. = not determined

TABLE
List of the tested bacteria except the E. coli/Shigella group

| | | PCR | Hybridisation |
|---|---|---|---|
| Species | Strain no. | detection | with probes |
| Buttiauxella agrestis | DSM 4586 | − | − |
| Cedecea davisae | DSM 4568 | − | − |
| Citrobacter amalonaticus | DSM 4593 | − | − |
| Citrobactef freundii | DSM 30040 | − | − |
| Citrobacter freundii | BC 6044 | − | − |
| Citrobacter koseri | DSM 4570 | − | − |
| Citrobacter koseri | DSM 4595 | − | − |
| Citrobacter koseri | BC 4962 | − | − |
| Edwartsiella tarda | DSM 30052 | − | − |
| Enterobacter aerogenes | DSM 30053 | − | − |
| Enterobacter aerogenes | BC 5895 | − | − |
| Enterobacter amnigenus | DSM 4486 | − | − |
| Enterobacter amnigenus | BC 7437 | − | − |
| Enterobacter amnigenus | BC 8794 | − | − |
| Enterobacter cloacae | DSM 30054 | − | − |
| Enterobacter cloacae | BC 2467 | − | − |
| Enterobacter cloacae | BC 8725 | − | − |
| Enterobacter gergoviae | BC 511 | − | − |
| Enterobacter gergoviae | BC 674 | − | − |
| Enterobacter intermedius | DSM 4581 | − | − |
| Enterobacter sakazakii | DSM 4485 | − | − |
| Erwinia carotovora subsp. carotovora | DSM 30168 | − | − |
| Escherichia blattae | NCTC 12127 | − | − |
| Escherichia hermannii | DSM 4560 | − | − |
| Escherichia hermannii | BC 8467 | − | − |
| Escherichia ferusonii | NCTC 12128 | (+) | − |
| Escherichia vulneris | DSM 4564 | − | − |
| Escherichia vulneris | BC 8793 | − | − |
| Hafnia alvei | BC 2154 | − | − |
| Klebsiella oxytoca | DSM 5175 | − | − |
| Klebsiella oxytoca | BC 2468 | − | − |
| Klebsiella planticola | DSM 4617 | − | − |
| Klebsiella pneumoniae | BC 5365 | − | − |
| Klebsiella pneumoniae subsp. pneumoniae | ATCC 13883 | − | − |
| Klebsiella pneumoniae subsp. pneumoniae | DSM 30102 | − | − |
| Klebsiella terrigena | DSM 2687 | − | − |
| Kluyvera ascorbata | DSM 4611 | − | − |
| Kluyvera sp. | BC 7440 | − | − |
| Morganella morganii subsp. morganii | DSM 30164 | − | − |
| Pantoea agglomerans | DSM 3493 | − | − |
| Pantoea agglomerans | BC 6043 | − | − |
| Pentoea agglomerans | BC 8600 | − | − |
| Pantoea spp. | BC 8669 | − | − |
| Pantoea spp. | BC 8726 | − | − |
| Proteus mirabilis | DSM 788 | − | − |
| Proteus rettgeri | DSM 1131 | − | − |
| Providencia stuartii | DSM 4539 | − | − |
| Rahnella aquatilis | DSM 4594 | − | − |
| Salmonella bongori V | BC 5695 | − | − |
| Salmonella bongori V | BC 7952 | − | − |
| Salmonella enterica I | BC 7751 | − | − |
| Salmonella enterica II | BC 5677 | − | − |
| Salmonella enterica IIIa | BC 5241 | − | − |
| Salmonella enterica IIIa | BC 5249 | − | − |
| Salmonella enterica IIIb | BC 7937 | − | − |
| Salmonella enterica IIIb | BC 7942 | − | − |
| Salmonella enterica IV | BC 7759 | − | − |
| Salmonella enterica VI | BC 7762 | − | − |
| Serratia marcescens | BC 677 | − | − |
| Serratia marcescens | DSM 1636 | − | − |
| Serratia odorifera | BC 678 | − | − |
| Serratia spp. | BC 1139 | − | − |
| Yersinia enterocolytica | DSM 4780 | − | − |
| Yersinia pseudotuberculosis | DSM 8992 | − | − |
| Yokenella regensburgei | DSM 5079 | − | − |
| Acinetobacter sp. | DSM 590 | − | − |
| Aeromonas hydrophila subsp. hydrophila | DSM 6173 | − | − |
| Bacillus cereus | NCFB 827 | − | − |
| Bacillus stearothermophilus | DSM 1550 | − | − |
| Bacillus subtilis | DSM 1970 | − | − |
| Carnobacterium mobile | DSM 4848 | − | − |
| Clostridium acetobutylicum | DSM 1731 | − | − |
| Clostridium propionicum | DSM 1682 | − | − |
| Clostridium saccharolyticum | DSM 2544 | − | − |
| Comamonas testosteroni | DSM 1622 | − | − |
| Enterococcus faecalis | DSM 6134 | − | − |
| Flavobacterum sp. | ATCC 27551 | − | − |
| Haemophilus influenzae | DSM 4690 | − | − |
| Lactococcus lactis subsp. hordniea | DSM 20450 | − | − |
| Lactococcus raffinolactis | DSM 20443 | − | − |
| Moraxella catarrhalis | DSM 9143 | − | − |
| Pasteurella pneumotropica | BC 2891 | − | − |
| Pediococcus inopinatus | DSM 20285 | − | − |
| Pseudomonas aeruginosa | DSM 50071 | − | − |
| Pseudomonas cepacia | BC 3134 | − | − |
| Pseudomonas fluorescens | DSM 6290 | − | − |
| Sphingomonas paucimobilis | BC 8795 | − | − |
| Sphingomonas sp. | DSM 6014 | − | − |
| Staphylococcus aureus subsp. aureus | DSM 20491 | − | − |
| Stenotrophomonas maltophila | BC 8724 | − | − |
| Streptococcus thermophilus | BC 2148 | − | − |
| Vibrio alginolyticus | DSM 2171 | − | − |
| Vibrio fischeri | DSM 507 | − | − |
| Vibrio harveyi | DSM 6904 | − | − |
| Vibrio parahaemolyticus | DSM 2172 | − | − |

Differentiation of Slt Genes

A characteristic feature of the VTEC is the presence of one of the two genes SltI (Shiga-like toxin) or SltII or both genes. These genes are also known as vtx1 and vtx2. For the precise type classification of VTEC and EHEC strains, further differentiation can be made with regard to the presence of these genes or of variants of these genes. In this way important information for the propagation of these pathogenic E. coli strains and also for evolution can be obtained. In addition there are indications that the pathological potential for various SltI or SltII variants or for the occurrence of both genes varies.

For the differentiation between SltI and SltII genes the primers of category A or categories B+C can be used.

The PCR reaction 1) was prepared as follows: I)

Sample volume—1 μl

10×PCR buffer—2.5 μl

10 mM dNTP—0.25 μl

10 μM forwards primer

Category A—0.2 μl

10 μM backwards primer

Category A—0.2 μl

50 mM MgCl₂—0.75 μl

5 U/μl Taq polymerase—0.3 μl

Water—add. 25 μl

The above reaction mixture was firmly closed in 200 μl reaction vessels and incubated according to the following protocol in a PCR device.

95° C.—5 min.

92° C.—1 min.)

52° C.—1 min.×35)

72° C.—0.5 min.)

72° C.—5 min.

In the reaction mixture one forwards and one backwards primer of the category A (Tab. 1-9) was each used.

In a further PCR reaction II) the following mixture was prepared: II)

Sample volume—1 μl

10×PCR buffer—2.5 μl

10 mM dNTP—0.25 μl

10 μM forwards primer

Category B+C—0.2 μl

10 μM backwards primer

Category B+C—0.2 μl

50 mM MgCl₂—0.75 μl

5 U/μl Taq polymerase-0.3 μl

Water-add. 25 μl

The above reaction mixture was firmly closed in 200 μl reaction vessels and incubated according to the following protocol in a PCR device.

95° C.—5 min.

92° C.—1 min.

52° C.—1 min.×35)

72° C.—0.5 min.)

72° C.—5 min.

In the reaction mixture one forwards and one backwards primer of the categories B+C (Tab. 1-9) was each used.

The results of the PCR reactions are summarised in the following table. A positive result was obtained when an amplicon which produced a band in the magnitude of approx. 500-700 bp was amplified. This was rendered visible on an agarose gel coloured with ethidium bromide.

TABLE
Differentiation between Sltl and Sltll genes

| | | | | Genes | | PCR detection | |
|---|---|---|---|---|---|---|---|
| | | | | present | | Category | Category |
| | BC no. | Origin | Sero var. | Sltl | Sltll | A | B + C |
| 1 | 12502 | Full-cream milk | O138H8 | − | + | − | + |
| 2 | 12503 | Full-cream milk | O157H— | − | + | − | + |
| 3 | 12504 | Beef | O8H27 | − | + | − | + |
| 4 | 12505 | Raw milk | O17H— | − | + | − | + |
| 5 | 12506 | Minced beef | O22H— | + | + | + | + |
| 6 | 12507 | Nuremberger grilled sausage | O157H— | − | + | − | + |
| 7 | 12508 | Lamb | O84H21 | + | + | + | + |
| 8 | 12509 | Lamb | O7H— | + | + | + | + |
| 9 | 12510 | Lamb | OntH— | + | − | + | − |
| 10 | 12511 | Cheese from raw cow's milk | O23H15 | − | + | − | + |
| 11 | 12512 | Minced beef, raw material | O8H— | − | + | − | + |
| 12 | 12513 | Minced beef, raw material | O— raw form | + | + | + | + |
| | | | H23 | | | | |
| 13 | 12514 | Minced beef, raw material | O46H— | − | + | − | + |
| 14 | 12515 | Minced beef | O104H12 | + | − | + | − |
| 15 | 12516 | Minced beef | O74H— | − | + | − | + |
| 16 | 12517 | Minced beef, raw material | O62H8 | + | + | + | + |
| 17 | 12518 | Minced beef, raw material | O157H7 | − | + | − | + |
| 18 | 12519 | Beef paté | O91H— | − | + | − | + |
| 19 | 12520 | Minced beef, raw material | O22H— | − | + | − | + |
| 20 | 12521 | Onion smoked sausage | O65H— | + | − | + | − |
| | | spread | | | | | |
| 21 | 12522 | Minced beef | O8H— | − | + | − | + |
| 22 | 12523 | Mixed minced meat | O91H21 | + | + | + | + |
| 23 | 12524 | Minced beef, raw material | O113H4 | − | + | − | + |
| 24 | 12525 | Minced beef | O22H8 | + | + | + | + |
| 25 | 12526 | Minced beef, raw material | O113H4 | + | + | + | + |

The primers of the categories A resp. B+C are also to be used in order to amplify sub-types of the SltI (category A) and SltII (category B+C) genes as consensus primers. These sub-types can be differentiated with specific probes such as are listed for categories A, resp. B+C. For sub-types not currently known, the probes of these categories can be tested empirically and assigned to the sub-types. Due to the large number of probes, a positive-negative pattern is produced which is characteristic of the sub-types. In addition, the primers of the categories A and B+C facilitate the amplification and subsequent sequencing of the amplicons. Also, techniques can be applied, such as mass spectrometry, hybridisation on biochips, “branch migration inhibition” or other techniques which enable an SNP (Single Nucleotide Polymorphism) analysis and are known to the specialist.

Optimisation of an On-line PCR

With an on-line PCR simultaneous amplification and detection of the amplicon occur. Depending on the amplicon to be detected, 1-2 colour-marked probes are added to the PCR mixture.

The detection of the amplicon can then take place, for example, with the aid of a 5′ nuclease assay (TaqMan probes), using molecular beacons, Scorpion assays or the previously described FRET technology.

In particular in the latter case it can only be determined empirically which of the probe pairs to be used are optimally suited. Often, the obtained fluorescence signal is too weak to give a reliable and reproducible result. In addition, in a complex PCR mixture probes can form dimers with other probes or primers, so that no on-line detection occurs.

With the detection of EHEC it can be advantageous to amplify both the Slt genes (−>VTEC) as well as the eae genes in a single multiplex PCR reaction (Slt genes+eae gene=EHEC) and then also to detect them simultaneously. In this case very precise matching of the reaction components is required. Through the consumption of the nucleotides, the amplification of one of two DNA target regions can be prevented. This signifies therefore that the amplification of a DNA is quenched by the amplification of another. It is therefore necessary to match all components of a PCR mixture to one another such that quenching does not occur.

This can also occur in that the primer concentration is limited. Here it must be considered that quenching is not a problem between the Slt genes, because the detection of only one Slt gene is adequate for the classification as VTEC. For this reason reduced amounts of SltI and SltII-specific primers can be added. The concentrations may be in the region of 300-200 nM per primer pair and PCR reaction. In contrast, the primer concentration of those for the eae gene should be higher (310-440 nM) in order to be able to also detect low eae DNA concentrations in the presence of higher Slt DNA concentrations.

A further method of preventing quenching due to the amplification of the Slt genes is to select an annealing temperature which is optimal for the eae-specific primers and less than optimal for the SltI and SltII-specific primers. Put more definitely, this temperature can be up to 5° C. above the optimum temperature for all Slt primers. The thermodynamic melting point can be regarded as the optimum temperature for primers.

The methods of preventing quenching can be used reciprocally if eae genes are present in excess in relation to SltI and SltII genes or quench the Slt detection for other reasons.

In the following, PCR conditions are shown which enable simultaneous amplification of the Slt and eae genes.

The PCR reaction is prepared as follows:

Sample volume—1 μl

10×PCR buffer—2 μl

Stabiliser—5.53 μl

10 mM dNTP—0.40 μl

10-4 μM forwards primer (primary sol.)

SEQ ID no. 1, 18, 68—0.2 μl

10-4 μM backwards primer

SEQ ID no. 6, 22, 73—0.2 μl

10 μM probes SEQ ID no. 93, 94, 95, 96, 97, 98, 9, 10, 35, 34

50 mM MgCl₂—1.6 μl

1 U/μl Taq polymerase—1 μl

Water—add. 20 μl

Temperature cycles in the Lightcycler:

92° C.—0 min.)

57° C.—1 min.×45)

72° C.—0.5 min.)

72° C.—5 min.

FIG. 4 shows the amplification of SltI and SltII genes by real-time PCR. Probes were used which facilitate the detection both of the SltI and the SltII genes. These were each coupled with the same fluorescent colouring (Lightcycler RED 640 and Fluorescein), so that the detection occurred in one channel (F2) only. It can be seen that with the amplification of the SltII genes, signal curves arise with amplitudes greater than 14. The signal curves of the SltI genes lie significantly lower. If both SltI and SltII genes occur, then the amplitude exhibits the highest level. It is therefore suitable as an indicator for the occurrence and the differentiation between the SltI and SltII genes.

It can also be seen from FIG. 4 that, depending on the application of various probes, the signal amplitude for the SltI genes varies. In the illustration the probes nos. 9+10 (strain nos. 1-10), nos. 95+96 (strain nos. 11-20), nos. 97+98 (strain nos. 21-30) and probes nos. 34+35 (strain nos. 1-30) were used together with the primers nos. 1+6 and 18+22. In addition, the oligonucleotides for the detection of the eae genes (see below) are present in the PCR mixture.

The eae gene was detected with probes which are coupled with the fluorescent colourings Lightcycler RED 705 and Fluorescein. Their detection occurred therefore in a different channel (F3) than that used for the Slt genes (F2). The probes nos. 93+94 and the primers nos. 68+73 were used for the eae detection. It can be seen in FIG. 5 that all eae-positive strains produce signal amplitudes which are greater than 5.

TABLE
Occurrence of pathogenicity genes with the
VTEC/EHEC strains used in the real-time PCR

| Strain no. in FIGS. 4, 5 | Sltl | Sltll | eae |
|---|---|---|---|
| 2, 12, 22 | − | + | + |
| 3, 13, 23 | − | + | + |
| 4, 14, 24 | + | + | − |
| 5, 15, 25 | − | + | − |
| 6, 16, 26 | + | − | + |
| 7, 17, 27 | + | − | + |
| 8, 18, 28 | + | − | + |
| 9, 19, 29 | + | − | + |
| 10, 20, 30 | + | − | + |

Strains in the same row in the above table are each identical (e.g. 2 = 12 = 22).

As object of this invention, oligonucleotides are provided which are particularly well suited to the detection of EHEC or VTEC. Within the number of these oligonucleotides there are some which are particularly well suited for this detection. They are summarised in the following table.

TABLE
Preferred oligonucleotide combinations for the detection of pathogenic E. coli

| Organisms to be | | |
|---|---|---|
| detected | Primers | Probes |
| VTEC | No. 1 + 6 + 18 + 22 | 9 + 10, 95 + 96, 97 + 98, 34 + 35 |
| VTEC | No. | 9 + 10, 95 + 96, 97 + 98, 34 + 35, |
| | 1 + 6 + 18 + 22 + 84 + 85 + 86 + 87 | 89 + 90 |
| EHEC | No. 1 + 6 + 18 + 22, 68 + 73 | 9 + 10, 95 + 96, 97 + 98, 34 + 35, |
| (see FIGS. 4 + 5) | | 93 + 94 |
| EHEC | No. 1 + 6 + 18 + 22, | 9 + 10, 95 + 96, 97 + 98, 34 + 35, |
| | 68 + 73 + 84 + 85 + 86 + 87 | 93 + 94, 89 + 90 |
| EHEC | No. 1 + 6 + 18 + 22 + 46 + 54 | 9 + 10, 95 + 96, 97 + 98, 34 + 35, |
| | | 60 + 61 |
| EHEC | No. 1 + 6 + 18 + 22, | 9 + 10, 95 + 96, 97 + 98, 34 + 35, |
| | 68 + 73 + 84 + 85 + 86 + 87 + 46 + 54 | 93 + 94, 89 + 90 + 60 + 61 |

Where a detection only occurs by visual indication of the amplicons in the agarose gel, the probes from the above table can be left out of the multiplex mixture.

TABLE
Optimisation of the real-time EHEC PCR

| Problem | Solution |
|---|---|
| Specification as EHEC | Simultaneous amplification of the Sltl/ll genes and an eae gene |
| | or detection in two PCR steps, where necessary. |
| | Detection of the species Escherichia coli in addition to the |
| | pathogenicity genes. |
| Specification as EHEC | Simultaneous amplification of the Sltl/ll genes and of the hlyA |
| | gene or detection in two PCR steps, where necessary. |
| | Detection of the species Escherichia coli in addition to the |
| | pathogenicity genes. |
| Specification as EHEC | Simultaneous amplification of the Sltl/ll genes and of the eae |
| | gene and of the hlyA gene or detection in three PCR steps, |
| | where necessary. |
| | Detection of the species Escherichia coli in addition to the |
| | pathogenicity genes. |
| Various Slt genes are | Sltl and Sltll genes can be differentiated by the curve traces and |
| detected with the same | the height of the amplitude. Further differentiation possible |
| fluorescent colouring | through melting curve analysis. |
| The simultaneous | Primers are limited. |
| amplification of the Slt and | |
| eae and/or hylA genes is | |
| quenched | |
| The amplification of the Slt | Annealing temperatures of the primers and/or probes are |
| and eae and/or hlyA genes | optimally selected with regard to quenching. |
| is quenched | |
| The amplification of the Slt | Selection of the probes and primers reduces quenching |
| and eae and/or hlyA genes | significantly. The amplification efficiency is decisively influenced |
| is quenched | by these oligonucleotides. Therefore, the primers and probes |
| | were matched harmoniously with one another. |
| The signal level for probes | Testing of a large number of probes/probe pairs and empirical |
| is too low | selection of the best probes. |

1. Method for the detection of EHEC bacteria in a sample, comprising the step:
Detection of the occurrence of a nucleic acid sequence from the slt locus in combination with a sequence from the eae locus and/or the hlya locus in the sample.

2. Method according to claim 1, characterised in that the detection includes at least one PCR.

3. Method according to one of the claims 1 or 2, characterised in that for the detection at least one oligonucleotide is used comprising at least one sequence selected from one of the SEQ ID numbers 1-83 and 93-98 and derivatives of them.

4. Method according to one of the claims 1-3, characterised in that at least one oligonucleotide is used comprising at least one sequence selected from one of the SEQ ID numbers 1-45 and 95-98 or derivatives of them (sequences of categories A-C) and at least one oligonucleotide comprising at least one sequence selected from one of the SEQ ID numbers 46-83 and 93 and 94 and derivatives of them (sequences of categories D and E).

5. Method according to one of the claims 1-4, characterised in that a forwards primer with a backwards primer from one of the categories A-C is combined with a forwards primer and a backwards primer from one of the categories D and E.

6. Method according to one of the claims 1-5, characterised in that an additional oligonucleotide is used comprising at least one sequence selected from one of the SEQ ID numbers 84-92 and derivatives of them (sequences of the category F).

7. Method according to one of the claims 1-6, characterised in that several oligonucleotides are used in the scope of a multiplex PCR or in at least two separate sequential PCRs.

8. Method according to one of the claims 1-7, characterised in that the detection includes bringing into contact the nucleic acid from the sample, after its amplification where necessary, with a biochip containing the oligonucleotides for the detection of EHEC.

9. Method according one of the claims 1-8, characterised in that it comprises at least one further step selected from
amplification of the nucleic acid to be detected;
PCR amplification of the nucleic acid to be detected;
southern blot hybridisation of the nucleic acid to be detected with suitable probes, preferably selected from a nucleic acid comprising at least one sequence with one of the SEQ ID numbers 1-98;
ligase chain reaction with the nucleic acid to be detected; and
isothermal nucleic acid amplification of the nucleic acid to be detected.

10. Method according to one of the claims 1-9, characterised in that the detection comprises an on-line detection of obtained amplicons.

11. Method according to one of the claims 1-10, characterised in that the amplification and/or detection of the nucleic acid to be detected occurs on a biochip.

12. Oligonucleotide for the detection of EHEC bacteria, selected from one of the nucleic acids comprising at least one sequence with one of the SEQ ID numbers 1-98 or derivatives of it.

13. Combination of oligonucleotides, comprising at least one oligonucleotide comprising at least one sequence selected from one of the categories A-C and at least one oligonucleotide comprising at least one sequence selected from one of the categories D and E, preferably one sequence D and one sequence from E.

14. Combination according to claim 13, characterised in that it furthermore comprises an oligonucleotide comprising at least one sequence selected from the category F.

15. Kit for the detection of EHEC bacteria containing an oligonucleotide according to claim 12 or a combination according to one of the claims 13 or 14.

16. Application of an oligonucleotide according to claim 12 and/or a combination according to claim 13 or 14 for the detection of EHEC bacteria.


SEQUENCE LISTING (98 sequences)

SEQ ID NO: 1 - DNA, 18 bp, Escherichia coli
ctggggaaggttgagtag

SEQ ID NO: 2 - DNA, 20 bp, Escherichia coli
gtcctgcctgaytatcatgg

SEQ ID NO: 3 - DNA, 21 bp, Escherichia coli
acaagactctgttcgtgtagg

SEQ ID NO: 4 - DNA, 27 bp, Escherichia coli
aagaatttcttttgraagyrttaatgc

SEQ ID NO: 5 - DNA, 28 bp, Escherichia coli
aattctgggwagcgtggcattaatactg

SEQ ID NO: 6 - DNA, 20 bp, Escherichia coli
cccactttaactgtaaaggt

SEQ ID NO: 7 - DNA, 29 bp, Escherichia coli
cgtcatcattatattttgtatactccacc

SEQ ID NO: 8 - DNA, 22 bp, Escherichia coli
cacttgctgaaaaaaatgaaag

SEQ ID NO: 9 - DNA, 26 bp, Escherichia coli
agcgtggcattaatactgaattgtca

SEQ ID NO: 10 - DNA, 25 bp, Escherichia coli
atcatgcatcgcgagttgccagaat

SEQ ID NO: 11 - DNA, 25 bp, Escherichia coli
atcatgcatcgcgagttgccagaat

SEQ ID NO: 12 - DNA, 35 bp, Escherichia coli
ttcgtgtwggaagaatttcttttgraagyrttaat

SEQ ID NO: 13 - DNA, 33 bp, Escherichia coli
atgagtttccttctatgtgyccggyagatggaa

SEQ ID NO: 14 - DNA, 37 bp, Escherichia coli
tccgtgggattacgcacaataaaatatttgtgggatt

SEQ ID NO: 15 - DNA, 32 bp, Escherichia coli
aaayattattaatagctgcatcrctttcattt

SEQ ID NO: 16 - DNA, 34 bp, Escherichia coli
ttcagcaagtgygctggckrcgccwgattctgta

SEQ ID NO: 17 - DNA, 33 bp, Escherichia coli
actggraaggtggagtatacaaaatataatgat

SEQ ID NO: 18 - DNA, 19 bp, Escherichia coli
ggcactgtctgaaactgct

SEQ ID NO: 19 - DNA, 20 bp, Escherichia coli
gaaactgctcctgtktatac

SEQ ID NO: 20 - DNA, 19 bp, Escherichia coli
gatgacrccggragamgtg

SEQ ID NO: 21 - DNA, 27 bp, Escherichia coli
ctgaactgggggmgaatcagcaatgtg

SEQ ID NO: 22 - DNA, 18 bp, Escherichia coli
ygccattgcattaacaga

SEQ ID NO: 23 - DNA, 23 bp, Escherichia coli
gcwgckgtattactttcccataa

SEQ ID NO: 24 - DNA, 32 bp, Escherichia coli
ggcctgtcgccagttatctgacattctggttg

SEQ ID NO: 25 - DNA, 32 bp, Escherichia coli; identical to SEQ ID NO: 24

SEQ ID NO: 26 - DNA, 19 bp, Escherichia coli
ggcgctgtctgaggcatct

SEQ ID NO: 27 - DNA, 20 bp, Escherichia coli
gaggcatctccgctttatac

SEQ ID NO: 28 - DNA, 19 bp, Escherichia coli
aatgacggctcaggatgtt

SEQ ID NO: 29 - DNA, 27 bp, Escherichia coli
ctgaactggggaagaataagtaatgtt

SEQ ID NO: 30 - DNA, 30 bp, Escherichia coli
gcagcgattgtattcgcttcccacaaaaca

SEQ ID NO: 31 - DNA, 32 bp, Escherichia coli
gccctgtctccaacaatctggcattctgtttt

SEQ ID NO: 32 - DNA, 21 bp, Escherichia coli
ctgtttttggctcacggaacg

SEQ ID NO: 33 - DNA, 22 bp, Escherichia coli
cgccatggaattagcagaaaag

SEQ ID NO: 34 - DNA, 21 bp, Escherichia coli
ccccagttcagwgtgaggtcc

SEQ ID NO: 35 - DNA, 21 bp, Escherichia coli
ccggaagcacattgctgattc

SEQ ID NO: 36 - DNA, 34 bp, Escherichia coli
gaatatcctttaataatatatcagcgatactkgg

SEQ ID NO: 37 - DNA, 33 bp, Escherichia coli
wgtggcsgttatactgaattgycatcatcaggg

SEQ ID NO: 38 - DNA, 28 bp, Escherichia coli
cgttcygttcgckccgtgaatgaagaka

SEQ ID NO: 39 - DNA, 32 bp, Escherichia coli
caaccagaatgtcagataactggcgacaggcc

SEQ ID NO: 40 - DNA, 21 bp, Escherichia coli
ccccagttcagggtaaggtca

SEQ ID NO: 41 - DNA, 21 bp, Escherichia coli
ctggaagaacattacttattc

SEQ ID NO: 42 - DNA, 35 bp, Escherichia coli
aggatatcttttaatagtctttctgcgattctcgg

SEQ ID NO: 43 - DNA, 33 bp, Escherichia coli
tgttgcggtcatccttaattgccactcaaccgg

SEQ ID NO: 44 - DNA, 29 bp, Escherichia coli
ttattcagttcgttccgtgagccaaaaac

SEQ ID NO: 45 - DNA, 32 bp, Escherichia coli
aaaacagaatgccagattgttggagacagggc

SEQ ID NO: 46 - DNA, 20 bp, Escherichia coli
  variant (9): n = Inosin
catgctgcntttttagaaga

SEQ ID NO: 47 - DNA, 20 bp, Escherichia coli
catgctgcrtttttagaaga

SEQ ID NO: 48 - DNA, 24 bp, Escherichia coli
  variant (9): n = Inosin
catgctgcntttttagaagactct

SEQ ID NO: 49 - DNA, 24 bp, Escherichia coli
catgctgcrtttttagaagactct

SEQ ID NO: 50 - DNA, 24 bp, Escherichia coli
aatgaatgggaaaaggagcatggc

SEQ ID NO: 51 - DNA, 23 bp, Escherichia coli
ctctctgtctttgcttgctgatt

SEQ ID NO: 52 - DNA, 30 bp, Escherichia coli
ctcgtcagcatgcagtagaaagagcagtcg

SEQ ID NO: 53 - DNA, 32 bp, Escherichia coli
cattgggatgagaagatcggtgaacttgcagg

SEQ ID NO: 54 - DNA, 21 bp, Escherichia coli
cgtctttatctccgagytcag

SEQ ID NO: 55 - DNA, 25 bp, Escherichia coli
acatcgtctttatctccgagytcag

SEQ ID NO: 56 - DNA, 32 bp, Escherichia coli
tttaccaacatccgtcttattataagatacgg

SEQ ID NO: 57 - DNA, 22 bp, Escherichia coli
ccttcaccagcaaatacttctg

SEQ ID NO: 58 - DNA, 22 bp, Escherichia coli
tgagcctgctccagaataaacc

SEQ ID NO: 59 - DNA, 23 bp, Escherichia coli
tcaattttgaataatcatataca

SEQ ID NO: 60 - DNA, 40 bp, Escherichia coli
agagaaagaaaacagagtggtaaatatgaatatatgacat

SEQ ID NO: 61 - DNA, 38 bp, Escherichia coli
tcttattgtaaatggtaaggatacatggtctgtaaaag

SEQ ID NO: 62 - DNA, 41 bp, Escherichia coli
gggaccatagacctttcaacaggtaatgtatcaagtgtttt

SEQ ID NO: 63 - DNA, 37 bp, Escherichia coli
acatttataacaccaacatttaccccaggagaagaag

SEQ ID NO: 64 - DNA, 42 bp, Escherichia coli
ggcatatattaattatctggaaaatggagggcttttagaggc

SEQ ID NO: 65 - DNA, 37 bp, Escherichia coli
caaccgaaggagtttacacaacaagtgtttgatcctc

SEQ ID NO: 66 - DNA, 35 bp, Escherichia coli
cattgggatgagaagatcggtgaacttgcaggcat

SEQ ID NO: 67 - DNA, 36 bp, Escherichia coli
aacccgtaatgctgatcgcagtcagagtggtaaggc

SEQ ID NO: 68 - DNA, 21 bp, Escherichia coli
ggcctggttacaacattatgg

SEQ ID NO: 69 - DNA, 25 bp, Escherichia coli
acgcgaaagataccgctcttggtat

SEQ ID NO: 70 - DNA, 21 bp, Escherichia coli
ccaggcttcgtcacagttgca

SEQ ID NO: 71 - DNA, 24 bp, Escherichia coli
ggaacggcagaggttaatctgcag

SEQ ID NO: 72 - DNA, 26 bp, Escherichia coli
agtggtaataactttgacggtagttc

SEQ ID NO: 73 - DNA, 18 bp, Escherichia coli
atccccatcgtcaccaga

SEQ ID NO: 74 - DNA, 21 bp, Escherichia coli
aacattatcaccataatactg

SEQ ID NO: 75 - DNA, 23 bp, Escherichia coli
tagtttacaccaacggtcgccgc

SEQ ID NO: 76 - DNA, 21 bp, Escherichia coli
cattacccgtaccatgacggt

SEQ ID NO: 77 - DNA, 27 bp, Escherichia coli
cggaactgcattgagtaaaggagatca

SEQ ID NO: 78 - DNA, 31 bp, Escherichia coli
tccagtgaactaccgtcaaagttatyaccac

SEQ ID NO: 79 - DNA, 31 bp, Escherichia coli; identical to SEQ ID NO: 78

SEQ ID NO: 80 - DNA, 28 bp, Escherichia coli
atgttgggctataacgtcttcattgatc

SEQ ID NO: 81 - DNA, 26 bp, Escherichia coli
aggatttttctggtgataatacccgt

SEQ ID NO: 82 - DNA, 42 bp, Escherichia coli
aggtattggtggcgaatactggcgagactatttcaaaagtag

SEQ ID NO: 83 - DNA, 41 bp, Escherichia coli
ttaacggctatttccgcatgagcggctggcatgagtcatac

SEQ ID NO: 84 - DNA, 22 bp, Escherichia coli
cgggtcaggtaattgcacagta

SEQ ID NO: 85 - DNA, 22 bp, Escherichia coli
cgggtcaggtgattgcacagta

SEQ ID NO: 86 - DNA, 22 bp, Escherichia coli
cgggtcaggtgattgcacaata

SEQ ID NO: 87 - DNA, 22 bp, Escherichia coli
cgggtcaggtaattgcacaata

SEQ ID NO: 88 - DNA, 22 bp, Escherichia coli
gcaacagttcagcaaagtccat

SEQ ID NO: 89 - DNA, 21 bp, Escherichia coli
cggtgaagccaccgacatcgt

SEQ ID NO: 90 - DNA, 24 bp, Escherichia coli
tggcaggttccggccttcactctc

SEQ ID NO: 91 - DNA, 17 bp, Escherichia coli
aagccaccgacatcgtg

SEQ ID NO: 92 - DNA, 17 bp, Escherichia coli
aagccactgacatcgtg

SEQ ID NO: 93 - DNA, 31 bp, Escherichia coli; identical to SEQ ID NO: 78

SEQ ID NO: 94 - DNA, 37 bp, Escherichia coli
ccagcatkttttcggaatcatagaacggtaataagaa

SEQ ID NO: 95 - DNA, 27 bp, Escherichia coli
attaayrcttycaaaagaaattcttcc

SEQ ID NO: 96 - DNA, 28 bp, Escherichia coli
cagtattaatgccacgctwcccagaatt

SEQ ID NO: 97 - DNA, 25 bp, Escherichia coli
ccttctatgtgyccggyagatggaa

SEQ ID NO: 98 - DNA, 20 bp, Escherichia coli
tscgtgggattacgcacaat